(19) United States
(12) Patent Application Publication
(10) Pub. No.: US 2021/0253550 A1
(43) Pub. Date: Aug. 19, 2021

(54) THERAPEUTIC COMPOUNDS AND COMPOSITIONS

(71) Applicant: eXIthera Pharmaceuticals, Inc., Westborough, MA (US)

(72) Inventors: Neil J. Hayward, Westborough, MA (US); Bertrand L. Chenard, Waterford, CT (US); Yuelian Xu, East Haven, CT (US); Roberta L. Dorow, Kalamazoo, MI (US); Michael E. Matison, Middleville, MI (US); Alexander Kolchinski, Winchester, MA (US); Richard Fornicola, Littleton, MA (US)

(21) Appl. No.: 17/245,691

(22) Filed: Apr. 30, 2021

Related U.S. Application Data
(63) Continuation of application No. PCT/US2019/058898, filed on Oct. 30, 2019.
(60) Provisional application No. 62/752,510, filed on Oct. 30, 2018.

(51) Int. Cl. C07D 401/06; A61P 7/02; A61L 33/04; A61L 33/00

(52) U.S. Cl. CPC C07D 401/06; A61P 7/02; A61L 2300/606; A61L 33/0041; C07B 2200/13; A61L 33/04

(57) ABSTRACT

Provided herein are compounds and compositions that inhibit Factor XIa or kallikrein and methods of using these compounds and compositions.

### CROSS-REFERENCE TO RELATED APPLICATIONS

This application is a continuation of International Application No. PCT/US2019/058898 filed Oct. 30, 2019, which claims benefit of and priority to U.S. Ser. No. 62/752,510 filed Oct. 30, 2018, each of which is incorporated herein by reference in its entirety.

### BACKGROUND

Blood coagulation is the first line of defense against blood loss following injury. The blood coagulation “cascade” involves a number of circulating serine protease zymogens, regulatory cofactors and inhibitors. Each enzyme, once generated from its zymogen, specifically cleaves the next zymogen in the cascade to produce an active protease. This process is repeated until finally thrombin cleaves the fibrinopeptides from fibrinogen to produce fibrin that polymerizes to form a blood clot. Although efficient clotting limits the loss of blood at a site of trauma, it also poses the risk of systemic coagulation resulting in massive thrombosis. Under normal circumstances, hemostasis maintains a balance between clot formation (coagulation) and clot dissolution (fibrinolysis). However, in certain disease states such as acute myocardial infarction and unstable angina, the rupture of an established atherosclerotic plaque results in abnormal thrombus formation in the coronary arterial vasculature.

Diseases that stem from blood coagulation, such as myocardial infarction, unstable angina, atrial fibrillation, stroke, pulmonary embolism, and deep vein thrombosis, are among the leading causes of death in developed countries. Current anticoagulant therapies, such as injectable unfractionated and low molecular weight (LMW) heparin and orally administered warfarin (coumadin), carry the risk of bleeding episodes and display patient-to-patient variability that results in the need for close monitoring and titration of therapeutic doses. Consequently, there is a large medical need for novel anticoagulation drugs that lack some or all of the side effects of currently available drugs.

Factor XIa is an attractive therapeutic target involved in the pathway associated with these diseases. Increased levels of Factor XIa or Factor XIa activity have been observed in several thromboembolic disorders, including venous thrombosis (Meijers et al., N. Engl. J. Med. 342:696, 2000), acute myocardial infarction (Minnema et al., Arterioscler Thromb Vasc Biol 20:2489, 2000), acute coronary syndrome (Butenas et al., Thromb Haemost 99:142, 2008), coronary artery disease (Butenas et al., Thromb Haemost 99:142, 2008), chronic obstructive pulmonary disease (Jankowski et al., Thromb Res 127:242, 2011), aortic stenosis (Blood Coagul Fibrinolysis, 22:473, 2011), acute cerebrovascular ischemia (Undas et al., Eur J Clin Invest, 42:123, 2012), and systolic heart failure due to ischemic cardiomyopathy (Zabcyk et al., Pol Arch Med Wewn. 120:334, 2010). Patients that lack Factor XI because of a genetic Factor XI deficiency exhibit few, if any, ischemic strokes (Salomon et al., Blood, 111:4113, 2008). At the same time, loss of Factor XIa activity, which leaves one of the pathways that initiate coagulation intact, does not disrupt hemostasis. In humans, Factor XI deficiency can result in a mild-to-moderate bleeding disorder, especially in tissues with high levels of local fibrinolytic activity, such as the urinary tract, nose, oral cavity, and tonsils. Moreover, hemostasis is nearly normal in Factor XI-deficient mice (Gailani, Blood Coagul Fibrinolysis, 8:134, 1997). Furthermore, inhibition of Factor XI has also been found to attenuate arterial hypertension and other diseases and dysfunctions, including vascular inflammation (Kossmann et al. Sci. Transl. Med. 9, eaah4923 (2017)).

Consequently, compounds that inhibit Factor XIa have the potential to prevent or treat a wide range of disorders while avoiding the side effects and therapeutic challenges that plague drugs that inhibit other components of the coagulation pathway. Moreover, due to the limited efficacy and adverse side effects of some current therapeutics for the inhibition of undesirable thrombosis (e.g., deep vein thrombosis, hepatic vein thrombosis, and stroke), improved compounds and methods (e.g., those associated with Factor XIa) are needed for preventing or treating undesirable thrombosis.

Another therapeutic target is the enzyme kallikrein. Human plasma kallikrein is a serine protease that may be responsible for activating several downstream factors (e.g., bradykinin and plasmin) that are critical for coagulation and control of e.g., blood pressure, inflammation, and pain. Kallikreins are expressed e.g., in the prostate, epidermis, and the central nervous system (CNS) and may participate in e.g., the regulation of semen liquefaction, cleavage of cellular adhesion proteins, and neuronal plasticity in the CNS. Moreover, kallikreins may be involved in tumorigenesis and the development of cancer and angioedema, e.g., hereditary angioedema. Overactivation of the kallikrein-kinin pathway can result in a number of disorders, including angioedema, e.g., hereditary angioedema (Schneider et al., J. Allergy Clin. Immunol. 120:2, 416, 2007). To date, there are limited treatment options for HAE (e.g., WO2003/076458).

New and improved crystalline forms of compounds that inhibit Factor XIa or kallikrein are needed. Crystalline forms of such compounds described herein are directed toward this end.

### SUMMARY

The present invention relates, in part, to novel forms (for example, certain crystalline forms described herein) of compounds that inhibit Factor XIa or kallikrein. Generally, a solid compound's efficacy as a drug can be affected by the properties of the solid it comprises.

In one aspect. the present invention is directed to a crystalline Compound 1:

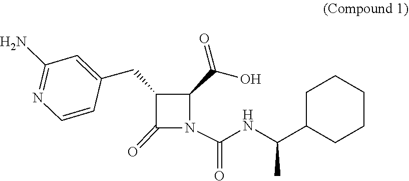 [SMILES: C[C@@H](NC(=O)N1C(=O)[C@H](Cc2ccnc(N)c2)[C@H]1C(=O)O)C1CCCCC1]

or a pharmaceutically acceptable salt thereof e.g., a hydrochloride salt of Compound 1. In some embodiments, a pharmaceutically acceptable salt of Compound 1 exists as a substantially pure crystalline solid form. In some embodiments, the pharmaceutically acceptable salt of Compound 1 is amorphous, e.g., the pharmaceutically acceptable salt of Compound 1 exists as a substantially amorphous solid form.

Thus in one aspect, provided herein is a crystalline pharmaceutically acceptable salt of Formula (I):

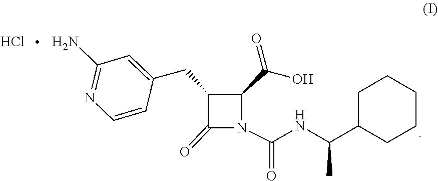 [SMILES: Cl ; C[C@@H](NC(=O)N1C(=O)[C@H](Cc2ccnc(N)c2)[C@H]1C(=O)O)C1CCCCC1]

The crystalline pharmaceutically acceptable salt of Formula (I) is a hydrochloride salt of Compound 1 and also referred to herein as Compound 1.HCl.

In some embodiments, physical or chemical parameters of a solid form of Compound 1 are evaluated from one or more of the following analytical techniques: X-ray powder diffraction (XRPD) analysis, single-crystal X-ray crystallography, thermogravimetric analysis (TGA), differential scanning calorimetry (DSC), nuclear magnetic resonance (NMR) spectroscopy, Karl Fisher (KF) titration, optical microscopy, or dynamic vapor sorption (DVS).

In some embodiments, a solid form is characterized and identified with parameters obtained from one or more of the aforementioned analytical methods:

X-ray diffraction patterns presented with degrees 2-theta (2θ) as the abscissa and peak intensity as the ordinate as determined by analysis with XRPD. These patterns are also referred to herein as XRPD patterns;

properties of the single-crystal structure of a solid form, e.g., unit cell, crystal system, and space group, as determined by single-crystal X-ray crystallography;

calculated XRPD patterns for a crystalline form as determined by data from single-crystal X-ray crystallography;

an endotherm specified by an onset temperature Tₒₙₛₑₜ that indicates a loss of solvent, a transformation from one crystalline form to another, or a melting point as determined by DSC performed at a specific ramp rate;

a value for weight loss as determined by TGA;

a value for weight gain at a temperature of 25° C. and a relative humidity between 2% and 95% as determined by DVS; and

an exemplary ¹H NMR spectrum of Compound 1.HCl dissolved in deuterated methanol (MeOD-d₄).

In some embodiments, a solid form is determined to be crystalline by the presence of sharp, distinct peaks found in the corresponding XRPD pattern.

In some embodiments, the crystalline pharmaceutically acceptable salt of Formula (I) has an XRPD pattern with characteristic peaks between and including the following values of 2θ in degrees: 7.4 to 7.8, 13.3 to 13.7, 14.3 to 14.7, 15.2 to 15.6, 16.3 to 16.7, 17.2 to 17.6, 18.8 to 19.2, 20.2 to 20.6, 23.5 to 23.9, and 26.7 to 27.1. In some embodiments, the crystalline pharmaceutically acceptable salt of Formula (I) has an XRPD pattern with characteristic peaks at the following values of 2θ in degrees: 7.6, 13.5, 14.5, 15.4, 16.5, 17.4, 19.0, 20.4, 23.7, and 26.9. In some embodiments, the crystalline pharmaceutically acceptable salt of Formula (I) has an XRPD pattern with characteristic peaks between and including the following values of 2θ in degrees: 7.4 to 7.8, 14.3 to 14.7, 16.3 to 16.7, 18.8 to 19.2, and 20.2 to 20.6. In some embodiments, the crystalline pharmaceutically acceptable salt of Formula (I) has an XRPD pattern with characteristic peaks at the following values of 2θ in degrees: 7.6, 14.5, 16.5, 19.0, and 20.4. In some embodiments, the crystalline pharmaceutically acceptable salt of Formula (I) has an XRPD pattern substantially as depicted in FIG. 1. In some embodiments, the crystalline pharmaceutically acceptable salt of Formula (I) has an XRPD pattern substantially as depicted in FIG. 26.

In some embodiments, the crystalline pharmaceutically acceptable salt of Formula (I) melts at a Tₒₙₛₑₜ from about 178° C. to about 192° C. as determined by DSC at a ramp rate of 10° C./min. In some embodiments, the crystalline pharmaceutically acceptable salt of Formula (I) has a DSC thermogram substantially as depicted in FIG. 6.

In an aspect, described herein is an amorphous pharmaceutically acceptable salt of Formula (I)

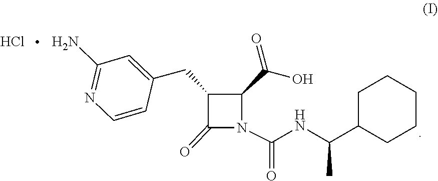 [SMILES: Cl ; C[C@@H](NC(=O)N1C(=O)[C@H](Cc2ccnc(N)c2)[C@H]1C(=O)O)C1CCCCC1]

In some embodiments, the amorphous pharmaceutically acceptable salt of Formula (I) has an endotherm at a Tₒₙₛₑₜ from about 95° C. to about 105° C. as determined by DSC at a ramp rate of 10° C./min. In some embodiments, the amorphous pharmaceutically acceptable salt of Formula (I), has a DSC thermogram substantially as depicted in FIG. 14. In some embodiments, the amorphous pharmaceutically acceptable salt of Formula (I), when subjected to a temperature of about 140° C., transforms into the crystalline compound of Formula (I) as indicated by DSC at a ramp rate of 10° C./min.

In an aspect, described herein is a pharmaceutical composition comprising a crystalline pharmaceutically acceptable salt of Formula (I) and a pharmaceutically acceptable excipient.

In an aspect, described herein is a pharmaceutical composition comprising an amorphous pharmaceutically acceptable salt of Formula (I) and a pharmaceutically acceptable excipient.

In an aspect, described herein is a compound of Formula (I) or pharmaceutical composition of Formula (I), for use in treating deep vein thrombosis in a subject that has suffered an ischemic event, comprising administering to the subject an effective amount of a compound of Formula (I) or pharmaceutical composition of Formula (I).

In an aspect, described herein is a compound of Formula (I) or pharmaceutical composition of Formula (I), for use in treating a subject that has edema, comprising administering to the subject an effective amount of a compound of Formula (I) or pharmaceutical composition of Formula (I).

In an aspect, provided herein is a method of treating a thromboembolic disorder in a subject in need thereof, the method comprising administering to the subject an effective amount of a compound represented by

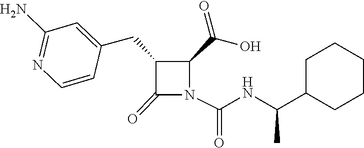 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In an aspect, provided herein is a method of reducing the risk of a thromboembolic disorder in a subject in need thereof, the method comprising administering to the subject an effective amount of a compound represented by

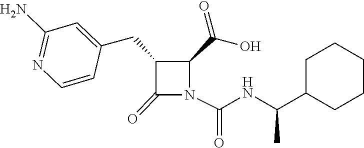 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In an aspect, provided herein is a method of prophylaxis of a thromboembolic disorder in a subject in need thereof, the method comprising administering to the subject an effective amount of a compound represented by

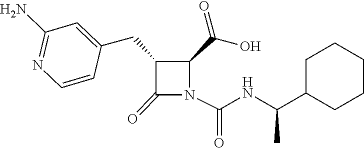 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In some embodiments of the methods provided herein, the artificial surface is in contact with blood in the subject's circulatory system. In some embodiments, the artificial surface is an implantable device, a dialysis catheter, a cardiopulmonary bypass circuit, an artificial heart valve, a ventricular assist device, a small caliber graft, a central venous catheter, or an extracorporeal membrane oxygenation (ECMO) apparatus. In some embodiments, the artificial surface causes or is associated with the thromboembolic disorder. In some embodiments, the thromboembolic disorder is a venous thromboembolism, deep vein thrombosis, or pulmonary embolism. In some embodiments, the thromboembolic disorder is a blood clot.

In some embodiments, the methods further comprise conditioning the artificial surface with a separate dose of the compound or pharmaceutically acceptable salt thereof, prior to contacting the artificial surface with blood in the circulatory system of the subject. In some embodiments, the methods further comprise conditioning the artificial surface with a separate dose of the compound or pharmaceutically acceptable salt thereof prior to or during administration of the compound or a pharmaceutically acceptable salt thereof to the subject. In some embodiments, the methods further comprise conditioning the artificial surface with a separate dose of the compound or pharmaceutically acceptable salt thereof prior to and during administration of the compound or a pharmaceutically acceptable salt thereof to the subject.

In an aspect, provided herein is a method of treating the blood of a subject in need thereof, the method comprising administering to the subject an effective amount of a compound represented by

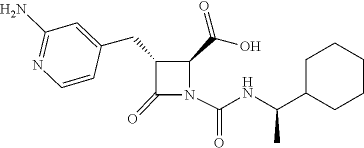 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In an aspect, provided herein is a method of maintaining the plasma level of a compound represented by

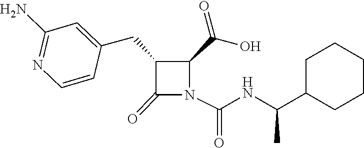 [SMILES: C[C@@H](NC(=O)N1C(=O)[C@H](Cc2ccnc(N)c2)[C@H]1C(=O)O)C1CCCCC1]

or a pharmaceutically acceptable salt thereof, in the blood of a subject in contact with an artificial surface, the method comprising:
(i) administering the compound or pharmaceutically acceptable salt thereof to the subject prior to or while contacting the artificial surface with the blood of the subject; and
(ii) conditioning an artificial surface with the compound or a pharmaceutically acceptable salt thereof prior to or while contacting the artificial surface with the blood of the subject; thereby maintaining the plasma level of the compound or a pharmaceutically acceptable salt thereof in the blood of the subject.

In some embodiments of the methods described herein, the compound, or a pharmaceutically acceptable salt thereof, maintains a constant activated partial thromboplastin time (aPTT) in the blood of the subject before and after contact with the artificial surface. In some embodiments, the compound or a pharmaceutically acceptable salt thereof is administered to the subject prior to and while contacting the artificial surface with the blood of the subject.

In some embodiments, the artificial surface is conditioned with the compound or a pharmaceutically acceptable salt thereof prior to and while contacting the artificial surface with the blood of the subject. In some embodiments, the method further prevents or reduces risk of a blood clot formation in the blood of the subject in contact with the artificial surface.

In some embodiments, the artificial surface is a cardiopulmonary bypass circuit. In some embodiments, the artificial surface is an extracorporeal membrane oxygenation (ECMO) apparatus. In some embodiments, the ECMO apparatus is venovenous ECMO apparatus or venoarterial ECMO apparatus.

In an aspect, provided herein is a method of preventing or reducing a risk of a thromboembolic disorder in a subject during or after a medical procedure, comprising:

(i) administering to the subject an effective amount of a compound represented by:

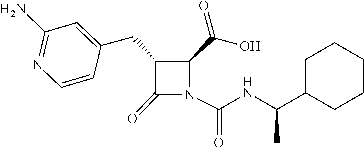 [SMILES: C[C@@H](NC(=O)N1C(=O)[C@H](Cc2ccnc(N)c2)[C@H]1C(=O)O)C1CCCCC1]

or pharmaceutically acceptable salt thereof, before, during, or after the medical procedure; and

(ii) contacting blood of the subject with an artificial surface;

thereby preventing or reducing the risk of the thromboembolic disorder during or after the medical procedure.

In some embodiments, the artificial surface is conditioned with the compound or pharmaceutically acceptable salt thereof prior to administration of the compound to the subject prior to, during, or after the medical procedure.

In some embodiments, the artificial surface is conditioned with a solution comprising the compound or a pharmaceutically acceptable salt thereof prior to administration of the compound or a pharmaceutically acceptable salt thereof to the subject prior to, during, or after the medical procedure. In some embodiments, the solution is a saline solution, Ringer's solution, or blood. In some embodiments, the solution further comprises blood. In some embodiments, the blood is acquired from the subject or a donor.

In some embodiments, the thromboembolic disorder is a blood clot.

In some embodiments, the medical procedure comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

In some embodiments, the medical procedure comprises a cardiopulmonary bypass. In some embodiments, the medical procedure comprises an oxygenation and pumping of blood via extracorporeal membrane oxygenation (ECMO). In some embodiments, the ECMO is venovenous ECMO or venoarterial ECMO.

In one aspect, the present invention is directed to a method of reducing the risk of stroke (e.g., ischemia, e.g., a transient ischemic event, large vessel acute ischemic stroke) in a subject that has suffered an ischemic event (e.g., a transient ischemic event), comprising administering to the subject an effective amount of Compound 1 or a pharmaceutically acceptable salt thereof, or of a composition described herein (e.g., a composition comprising Compound 1). In some embodiments, the administering reduces the risk of stroke (e.g., large vessel acute ischemic stroke) in a subject as compared to a subject who is not administered with the compound. In some embodiments, the administering reduces the risk of atrial fibrillation in a subject as compared to a subject who is not administered with the compound.

In one aspect, the present invention is directed to a method of reducing non-central nervous system systemic embolism (e.g., ischemia, e.g., a transient ischemic event) in a subject that has suffered an ischemic event (e.g., a transient ischemic event), comprising administering to the subject an effective amount of Compound 1 or a pharmaceutically acceptable salt thereof, or of a composition described herein (e.g., a composition comprising Compound 1). In some embodiments, the administering reduces non-central nervous system systemic embolism in a subject as compared to a subject who is not administered with the compound.

In one aspect, the present invention is directed to a method of treating deep vein thrombosis comprising administering to the subject that has suffered an ischemic event (e.g., a transient ischemic event), an effective amount of Compound 1 or a pharmaceutically acceptable salt thereof, or of a composition described herein (e.g., a composition comprising Compound 1).

In one aspect, the present invention is directed to a method of prophylaxis of deep vein thrombosis comprising administering to the subject that has suffered a deep vein thrombosis (e.g., a subject that has been previously treated for a deep vein thrombosis), an effective amount of Compound 1 or a pharmaceutically acceptable salt thereof, or of a composition described herein (e.g., a composition comprising Compound 1). In one aspect, the present invention is directed to a method of reducing the risk of recurrence of deep vein thrombosis comprising administering to the subject that has suffered a deep vein thrombosis (e.g., a subject that has been previously treated for a deep vein thrombosis), an effective amount of Compound 1 or a pharmaceutically acceptable salt thereof, or of a composition described herein (e.g., a composition comprising Compound 1). In some embodiments, the administering reduces the risk of recurrence of deep vein thrombosis in a subject as compared to a subject who is not administered with the compound.

In one aspect, the present invention is directed to a method of prophylaxis of venous thromboembolism, e.g., deep vein thrombosis or pulmonary embolism in a subject, comprising administering to the subject an effective amount of Compound 1 or a pharmaceutically acceptable salt thereof, or of a composition described herein (e.g., a composition comprising Compound 1). In some embodiments, the subject is undergoing surgery. In some embodiments, the subject is administered the compound, pharmaceutically acceptable salt thereof, or composition thereof before, during, or after surgery. In some embodiments, the subject is undergoing knee or hip replacement surgery. In some embodiments, the subject is undergoing orthopedic surgery. In some embodiments, the subject is undergoing lung surgery. In some embodiments, the subject is being treated for cancer, e.g., by surgery. In some embodiments, the subject is suffering from a chronic medical condition. In some embodiments, the venous thromboembolism is associated with cancer. In some embodiments, the compound, pharmaceutically acceptable salt thereof, or composition described herein is a primary agent in prophylaxis of the deep vein thrombosis or venous thromboembolism. In some embodiments, the compound, pharmaceutically acceptable salt thereof, or composition described herein is used as an extended therapy. In one aspect, the present invention is directed to a method of reducing the risk of venous thromboembolism, e.g., deep vein thrombosis or pulmonary embolism, in a subject, comprising administering to the subject an effective amount of Compound 1 or a pharmaceutically acceptable salt thereof, or of a composition described herein (e.g., a composition comprising Compound 1). In some embodiments, the subject is undergoing surgery. In some embodiments, the subject is administered the compound, pharmaceutically acceptable salt thereof, or composition thereof after surgery. In some embodiments, the subject is undergoing knee or hip replacement surgery. In some embodiments, the subject is undergoing orthopedic surgery. In some embodiments, the subject is undergoing lung surgery. In some embodiments, the subject is being treated for cancer, e.g., by surgery. In some embodiments, the subject is suffering from a chronic medical condition. In some embodiments, the thromboembolic disorder is associated with cancer. In some embodiments, the compound, pharmaceutically acceptable salt thereof, or composition described herein is a primary agent in reducing the risk of the thromboembolic disorder. In some embodiments, the compound, pharmaceutically acceptable salt thereof, or composition described herein is used as an extended therapy.

In one aspect, the present invention is directed to a method of reducing the risk of stroke (e.g., large vessel acute ischemic stroke) or systemic embolism in a subject in need thereof, comprising administering to the subject an effective amount of a compound described herein, e.g, Compound 1, or a pharmaceutically acceptable salt thereof, or a composition described herein, e.g., a composition comprising Compound 1. In some embodiments, the subject is suffering from atrial fibrillation (e.g., non-valvular atrial fibrillation). In some embodiments, the subject is suffering from a renal disorder (e.g., end-stage renal disease).

In one aspect, the present invention is directed to a method of prophylaxis of stroke (e.g., large vessel acute ischemic stroke) or systemic embolism in a subject in need thereof, comprising administering to the subject an effective amount of a compound described herein, e.g, Compound 1, or a pharmaceutically acceptable salt thereof, or a composition described herein, e.g., a composition comprising Compound 1. In some embodiments, the subject is suffering from atrial fibrillation (e.g., non-valvular atrial fibrillation). In some embodiments, the subject is suffering from a renal disorder (e.g., end-stage renal disease).

In one aspect, the present invention is directed to a method of reducing the risk of recurrence of pulmonary embolism (e.g., symptomatic pulmonary embolism) comprising administering to the subject that has suffered a pulmonary embolism (e.g., a subject that has been previously treated for a pulmonary embolism), an effective amount of Compound 1 or a pharmaceutically acceptable salt thereof, or of a composition described herein (e.g., a composition comprising Compound 1). In some embodiments, the administering reduces the risk of recurrence of pulmonary embolism in a subject as compared to a subject who is not administered with the compound.

In one aspect, the present invention is directed to a method of prophylaxis of pulmonary embolism in a subject that has suffered a pulmonary embolism (e.g., a subject that has been previously treated for a pulmonary embolism), comprising administering to the subject an effective amount of Compound 1 or a pharmaceutically acceptable salt thereof, or of a composition described herein (e.g., a composition comprising Compound 1).

In one aspect, the present invention is directed to a method of reducing the risk of recurrence of pulmonary embolism (e.g., symptomatic pulmonary embolism) comprising administering to the subject that has suffered a deep vein thrombosis (e.g., a subject that has been previously treated for a deep vein thrombosis), an effective amount of Compound 1 or a pharmaceutically acceptable salt thereof, or of a composition described herein (e.g., a composition comprising Compound 1). In some embodiments, the administering reduces the risk of recurrence of pulmonary embolism in a subject as compared to a subject who is not administered with the compound.

In one aspect, the present invention is directed to a method of prophylaxis of pulmonary embolism in a subject that has suffered a deep vein thrombosis (e.g., a subject that has been previously treated for a deep vein thrombosis), comprising administering to the subject an effective amount of Compound 1 or a pharmaceutically acceptable salt thereof, or of a composition described herein (e.g., a composition comprising Compound 1).

In one aspect, the present invention features a method of treating deep vein thrombosis in a subject that has been previously administered an anticoagulant, comprising administering to the subject an effective amount of Compound 1 or a pharmaceutically acceptable salt thereof, or of a composition described herein (e.g., a composition comprising Compound 1). In some embodiments, the anticoagulant was administered parenterally for 5-10 days.

In one aspect, the present invention features a method of treating a pulmonary embolism in a subject that has been previously administered an anticoagulant, comprising administering to the subject an effective amount of Compound 1 or a pharmaceutically acceptable salt thereof, or of a composition described herein (e.g., a composition comprising Compound 1). In some embodiments, the anticoagulant was administered parenterally for 5-10 days.

In one aspect, the present invention is directed to a method of treating a subject that has had an ischemic event (e.g., transient ischemia), comprising: administering Compound 1 or a pharmaceutically acceptable salt thereof, or of a composition described herein (e.g., a composition comprising Compound 1) to the subject. In some embodiments, the compound is administered to the subject within 24 hours or less, e.g., 12, 10, 9, 8, 7, 6 hours or less, after the onset of the ischemic event in the subject.

In one aspect, the present invention is directed to a method of treating a subject that has had an ischemic event (e.g., transient ischemia), comprising: administering Compound 1 or a pharmaceutically acceptable salt thereof, or of a composition described herein (e.g., a composition comprising Compound 1) to the subject. In some embodiments, the compound is administered to the subject within more than 2 hours to 12 hours, e.g., more than 2 hours to 10 hours or less, more than 2 hours to 8 hours or less, after the onset of the ischemic event in the subject.

In one aspect, the present invention is directed to a method of treating hypertension, e.g., arterial hypertension, in a subject, comprising administering to the subject an effective amount of Compound 1 or a pharmaceutically acceptable salt thereof, or of a composition described herein (e.g., a composition comprising Compound 1). In some embodiments, the hypertension, e.g., arterial hypertension, results in atherosclerosis. In some embodiments, the hypertension is pulmonary arterial hypertension.

In one aspect, the present invention is directed to a method of reducing the risk of hypertension, e.g., arterial hypertension, in a subject, comprising administering to the subject an effective amount of Compound 1 or a pharmaceutically acceptable salt thereof, or of a composition described herein (e.g., a composition comprising Compound 1). In some embodiments, the hypertension, e.g., arterial hypertension, results in atherosclerosis. In some embodiments, the hypertension is pulmonary arterial hypertension.

In one aspect, the present invention is directed to a method of prophylaxis of hypertension, e.g., arterial hypertension, in a subject, comprising administering to the subject an effective amount of Compound 1 or a pharmaceutically acceptable salt thereof, or of a composition described herein (e.g., a composition comprising Compound 1). In some embodiments, the hypertension, e.g., arterial hypertension, results in atherosclerosis. In some embodiments, the hypertension is pulmonary arterial hypertension.

In one aspect, the present invention is directed to a method of reducing inflammation in a subject, comprising administering to the subject an effective amount of Compound 1 or a pharmaceutically acceptable salt thereof, or of a composition described herein (e.g., a composition comprising Compound 1). In some embodiments, the inflammation is vascular inflammation. In some embodiments, the vascular inflammation is accompanied by atherosclerosis. In some embodiments, the vascular inflammation is accompanied by a thromboembolic disease in the subject. In some embodiments, the vascular inflammation is angiotensin II-induced vascular inflammation.

In one aspect, the present invention is directed to a method of preventing vascular leukocyte infiltration in a subject, comprising administering to the subject an effective amount of Compound 1 or a pharmaceutically acceptable salt thereof, or of a composition described herein (e.g., a composition comprising Compound 1).

In one aspect, the present invention is directed to a method of preventing angiotensin II-induced endothelial dysfunction in a subject, comprising administering to the subject an effective amount of Compound 1 or a pharmaceutically acceptable salt thereof, or of a composition described herein (e.g., a composition comprising Compound 1).

In one aspect, the present invention is directed to a method of preventing thrombin propagation in a subject, comprising administering to the subject an effective amount of Compound 1 or a pharmaceutically acceptable salt thereof, or of a composition described herein (e.g., a composition comprising Compound 1). In some embodiments, the thrombin propagation occurs on platelets.

In one aspect, the present invention is directed to a method of treating hypertension-associated renal dysfunction in a subject, comprising administering to the subject an effective amount of Compound 1 or a pharmaceutically acceptable salt thereof, or of a composition described herein (e.g., a composition comprising Compound 1).

In one aspect, the present invention is directed to a method of prophylaxis of hypertension-associated renal dysfunction in a subject, comprising administering to the subject an effective amount of Compound 1 or a pharmaceutically acceptable salt thereof, or of a composition described herein (e.g., a composition comprising Compound 1).

In one aspect, the present invention is directed to a method of reducing the risk of hypertension-associated renal dysfunction in a subject, comprising administering to the subject an effective amount of Compound 1 or a pharmaceutically acceptable salt thereof, or of a composition described herein (e.g., a composition comprising Compound 1).

In one aspect, the present invention is directed to a method of treating kidney fibrosis in a subject, comprising administering to the subject an effective amount of Compound 1 or a pharmaceutically acceptable salt thereof, or of a composition described herein (e.g., a composition comprising Compound 1).

In one aspect, the present invention is directed to a method of prophylaxis of kidney fibrosis in a subject, comprising administering to the subject an effective amount of Compound 1 or a pharmaceutically acceptable salt thereof, or of a composition described herein (e.g., a composition comprising Compound 1).

In one aspect, the present invention is directed to a method of reducing the risk of kidney fibrosis in a subject, comprising administering to the subject an effective amount of Compound 1 or a pharmaceutically acceptable salt thereof, or of a composition described herein (e.g., a composition comprising Compound 1).

In one aspect, the present invention is directed to a method of treating kidney injury in a subject, comprising administering to the subject an effective amount of Compound 1 or a pharmaceutically acceptable salt thereof, or of a composition described herein (e.g., a composition comprising Compound 1).

In one aspect, the present invention is directed to a method of prophylaxis of kidney injury in a subject, comprising administering to the subject an effective amount of Compound 1 or a pharmaceutically acceptable salt thereof, or of a composition described herein (e.g., a composition comprising Compound 1).

In one aspect, the present invention is directed to a method of reducing the risk of kidney injury in a subject, comprising administering to the subject an effective amount of Compound 1 or a pharmaceutically acceptable salt thereof, or of a composition described herein (e.g., a composition comprising Compound 1). In one aspect, the present invention is directed to a method of inhibiting Factor XIa in a subject, comprising administering to the subject that has suffered ischemia an effective amount of Compound 1 or a pharmaceutically acceptable salt thereof, or of a composition described herein (e.g., a composition comprising Compound 1). In some embodiments, the ischemia is coronary ischemia. In some embodiments, the subject is a mammal (e.g., a human). In some embodiments, the subject is undergoing surgery (e.g., knee replacement surgery or hip replacement surgery). In some embodiments, the ischemia is coronary ischemia. In some embodiments, the subject is a subject with non-valvular atrial fibrillation. In some embodiments, the subject has one or more of the following risk factors for stroke: a prior stroke (e.g., ischemic, unknown, hemorrhagic), transient ischemic attack, or non-CNS systemic embolism. In some embodiments, the subject has one or more of the following risk factors for stroke: 75 years or older of age, hypertension, heart failure or left ventricular ejection fraction (e.g., less than or equal to 35%), or diabetes mellitus.

In some embodiments, the compound is administered by oral or parenteral (e.g., intravenous) administration. In some embodiments, the compound is administered by oral administration. In some embodiments, the compound is administered by parenteral (e.g., intravenous) administration. In some embodiments, the compound is administered by subcutaneous administration.

In some embodiments, the compound is administered prior to an ischemic event (e.g., to a subject is at risk of an ischemic event).

In some embodiments, the compound is administered after an ischemic event (e.g., a transient ischemic event). In some embodiments, the compound is administered about 1, 2, 3, 4, 5, 6, 7, 8, 9, 10, 11, 12, 13, or 14 days or more after an ischemic event (e.g., a transient ischemic event). In some embodiments, the compound is administered about 1, 2, 3, 4, 5, 6, 7, or 8 weeks or more after an ischemic event (e.g., a transient ischemic event).

In some embodiments, the compound is administered in combination with an additional therapeutic agent. In some embodiments, the additional therapeutic agent is administered after administration of the compound. In some embodiments, the additional therapeutic agent is administered orally. In some embodiments, the additional therapeutic agent is administered at least 1, 2, 3, 4, 5, 6, 7, 8, 10, 12, 14, 16, 18, 20, or 24 hours or more after administration of the compound. In some embodiments, the additional therapeutic agent is administered at least 1, 2, 3, 4, 5, 6, 7, 14, 21, or 28 days or more after administration of the compound. In some embodiments, the additional therapeutic agent is administered about 1 day, about 2 days, about 3 days, about 4 days, about 5 days, about 6 days, about 7 days or more after administration of the compound.

In some embodiments, the additional therapeutic agent is administered chronically (e.g., for about 1 day, about 2 days, about 3 days, about 4 days, about 5 days, about 6 days, about 7 days, about 8 days, about 9 days, about 10 days, about 11 days, about 12 days, about 13 days, or about 14 days or more) after administration of the compound.

In some embodiments, the additional therapeutic agent treats a side effect (e.g., active pathological bleeding or severe hypersensitivity reactions (e.g., anaphylactic reactions), spinal and or epidural hematoma, gastrointestinal disorder (e.g., abdominal pain upper, dyspepsia, toothache), general disorders and administration site conditions (e.g., fatigue), infections and infestations (e.g., sinusitis, urinary tract infection), musculoskeletal and connective tissues disorders (e.g., back pain, osteoarthritis), respiratory, thoracic and mediastinal disorders (e.g., oropharyngeal pain), injury, poisoning, and procedural complications (e.g., wound secretion), musculoskeletal and connective tissues disorders (e.g., pain in extremity, muscle spasm), nervous system disorders (e.g., syncope), skin and subcutaneous tissue disorders (e.g., pruritus, blister), blood and lymphatic system disorders (e.g., agranulocytosis), gastrointestinal disorders (e.g., retroperitoneal hemorrhage), hepatobiliary disorders (e.g., jaundice, cholestasis, cytolytic hepatitis), immune system disorders (e.g., hypersensitivity, anaphylactic reaction, anaphylactic shock, angioedema), nervous system disorders (e.g., cerebral hemorrhage, subdural hematoma, epidural hematoma, hemiparesis), skin and subcutaneous tissue disorders (e.g., Stevens-Johnson syndrome).

In some embodiments, the additional therapeutic agent is a NSAID (e.g., aspirin or naproxen), platelet aggregation inhibitor (e.g., clopidogrel), or anticoagulant (e.g., warfarin or enoxaparin).

In some embodiments, the additional therapeutic agent results in an additive therapeutic effect. In some embodiments, the additional therapeutic agent results in a synergistic therapeutic effect.

In another aspect, the present invention features a method of modulating (e.g., inhibiting) Factor XIa in a patient. The method comprises the step of administering an effective amount of a compound described herein (e.g., Compound 1) or a pharmaceutically acceptable salt thereof, or of a composition described herein (e.g., a composition comprising Compound 1) to a patient in need thereof, thereby modulating (e.g., inhibiting) Factor XIa.

In another aspect, the present invention features a method of treating a subject in need thereof for a thromboembolic disorder. The method comprises administering to the subject an effective amount of a compound described herein (e.g., Compound 1) or a pharmaceutically acceptable salt thereof, or of a composition described herein (e.g., a composition comprising Compound 1). The thromboembolic disorder can be arterial cardiovascular thromboembolic disorders, arterial thrombosis, venous cardiovascular thromboembolic disorders, and thromboembolic disorders in the chambers of the heart; including unstable angina, an acute coronary syndrome, first myocardial infarction, recurrent myocardial infarction, ischemia (e.g., coronary ischemia, ischemic sudden death, or transient ischemic attack), stroke (e.g., large vessel acute ischemic stroke), atherosclerosis, peripheral occlusive arterial disease, venous thromboembolism, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (0 other procedures in which blood is exposed to an artificial surface that promotes thrombosis.

In another aspect, the present invention features a method of prophylaxis of a thromboembolic disorder in a subject. The method comprises administering to the subject an effective amount of a compound described herein (e.g., Compound 1) or a pharmaceutically acceptable salt thereof, or of a composition described herein (e.g., a composition comprising Compound 1). The thromboembolic disorder can be arterial cardiovascular thromboembolic disorders, arterial thrombosis, venous cardiovascular thromboembolic disorders, and thromboembolic disorders in the chambers of the heart; including unstable angina, an acute coronary syndrome, first myocardial infarction, recurrent myocardial infarction, ischemia (e.g., coronary ischemia, ischemic sudden death, or transient ischemic attack), stroke (e.g., large vessel acute ischemic stroke), atherosclerosis, peripheral occlusive arterial disease, venous thromboembolism, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (0 other procedures in which blood is exposed to an artificial surface that promotes thrombosis.

In another aspect, the present invention features a method of reducing the risk of a thromboembolic disorder in a subject. The method comprises administering to the subject an effective amount of a compound described herein (e.g., Compound 1) or a pharmaceutically acceptable salt thereof, or of a composition described herein (e.g., a composition comprising Compound 1). The thromboembolic disorder can be arterial cardiovascular thromboembolic disorders, arterial thrombosis, venous cardiovascular thromboembolic disorders, and thromboembolic disorders in the chambers of the heart; including unstable angina, an acute coronary syndrome, first myocardial infarction, recurrent myocardial infarction, ischemia (e.g., coronary ischemia, ischemic sudden death, or transient ischemic attack), stroke (e.g., large vessel acute ischemic stroke), atherosclerosis, peripheral occlusive arterial disease, venous thromboembolism, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (0 other procedures in which blood is exposed to an artificial surface that promotes thrombosis.

In one aspect, the present invention is directed to a method of treating end-stage renal disease in a subject, comprising administering to the subject an effective amount of Compound 1 or a pharmaceutically acceptable salt thereof, or of a composition described herein (e.g., a composition comprising Compound 1).

In one aspect, the present invention is directed to a method of prophylaxis of end-stage renal disease in a subject, comprising administering to the subject an effective amount of Compound 1 or a pharmaceutically acceptable salt thereof, or of a composition described herein (e.g., a composition comprising Compound 1).

In one aspect, the present invention is directed to a method of reducing the risk of end-stage renal disease in a subject, comprising administering to the subject an effective amount of Compound 1 or a pharmaceutically acceptable salt thereof, or of a composition described herein (e.g., a composition comprising Compound 1).

In another aspect, the present invention features a method of treating a thromboembolic disorder in a subject need thereof, the method comprising administering to the subject an effective amount of a compound described herein (e.g., Compound 1) or a pharmaceutically acceptable salt thereof, or of a composition described herein (e.g., a composition comprising Compound 1), wherein the subject is exposed to an artificial surface. In some embodiments, the artificial surface contacts the subject's blood. In some embodiments, the artificial surface is an extracorporeal surface. In some embodiments, the artificial surface is that of an implantable device, e.g., a mechanical valve. In some embodiments, the artificial surface is that of a dialysis catheter. In some embodiments, the artificial surface is that of a cardiopulmonary bypass circuit. In some embodiments, the artificial surface is that of an artificial heart valve. In some embodiments, the artificial surface is that of a ventricular assist device. In some embodiments, the artificial surface is that of a small caliber graft. In some embodiments, the artificial surface is that of a central venous catheter. In some embodiments, the artificial surface is that of an extracorporeal membrane oxygenation (ECMO) apparatus. In some embodiments, the artificial surface causes or is associated with the thromboembolic disorder. In some embodiments, the thromboembolic disorder is a venous thromboembolism. In some embodiments, the thromboembolic disorder is deep vein thrombosis. In some embodiments, the thromboembolic disorder is pulmonary embolism.

In another aspect, the present invention features a method of reducing the risk of a thromboembolic disorder in a subject need thereof, the method comprising administering to the subject an effective amount of a compound described herein (e.g., Compound 1) or a pharmaceutically acceptable salt thereof, or of a composition described herein (e.g., a composition comprising Compound 1), wherein the subject is exposed to an artificial surface. In some embodiments, the artificial surface contacts the subject's blood. In some embodiments, the artificial surface is an extracorporeal surface. In some embodiments, the artificial surface is that of an implantable device, e.g., a mechanical valve. In some embodiments, the artificial surface is that of a dialysis catheter. In some embodiments, the artificial surface is that of a cardiopulmonary bypass circuit. In some embodiments, the artificial surface is that of an artificial heart valve. In some embodiments, the artificial surface is that of a ventricular assist device. In some embodiments, the artificial surface is that of a small caliber graft. In some embodiments, the artificial surface is that of a central venous catheter. In some embodiments, the artificial surface is that of an extracorporeal membrane oxygenation (ECMO) apparatus. In some embodiments, the artificial surface causes or is associated with the thromboembolic disorder. In some embodiments, the thromboembolic disorder is a venous thromboembolism. In some embodiments, the thromboembolic disorder is deep vein thrombosis. In some embodiments, the thromboembolic disorder is pulmonary embolism.

In another aspect, the present invention features a method of prophylaxis of a thromboembolic disorder in a subject need thereof, the method comprising administering to the subject an effective amount of a compound described herein (e.g., Compound 1) or a pharmaceutically acceptable salt thereof, or of a composition described herein (e.g., a composition comprising Compound 1), wherein the subject is exposed to an artificial surface. In some embodiments, the artificial surface contacts the subject's blood. In some embodiments, the artificial surface is an extracorporeal surface. In some embodiments, the artificial surface is that of an implantable device, e.g., a mechanical valve. In some embodiments, the artificial surface is that of a dialysis catheter. In some embodiments, the artificial surface is that of a cardiopulmonary bypass circuit. In some embodiments, the artificial surface is that of an artificial heart valve. In some embodiments, the artificial surface is that of a ventricular assist device. In some embodiments, the artificial surface is that of a small caliber graft. In some embodiments, the artificial surface is that of a central venous catheter. In some embodiments, the artificial surface is that of an extracorporeal membrane oxygenation (ECMO) apparatus. In some embodiments, the artificial surface causes or is associated with the thromboembolic disorder. In some embodiments, the thromboembolic disorder is a venous thromboembolism. In some embodiments, the thromboembolic disorder is deep vein thrombosis. In some embodiments, the thromboembolic disorder is pulmonary embolism.

In another aspect, the present invention features a method of treating atrial fibrillation, in a subject in need thereof, the method comprising administering to the subject an effective amount of a compound described herein (e.g., Compound 1) or a pharmaceutically acceptable salt thereof, or of a composition described herein (e.g., a composition comprising Compound 1). In some embodiments, the subject is also in need of dialysis, e.g., renal dialysis. In some embodiments, the compound described herein is administered to the subject while the subject is undergoing dialysis. In some embodiments, the compound or pharmaceutically acceptable salt or composition is administered to the subject before or after receiving dialysis. In some embodiments, the patient has end-stage renal disease. In some embodiments, the subject is not in need of dialysis, e.g., renal dialysis. In some embodiments, the patient is at a high risk for bleeding. In some embodiments, the atrial fibrillation is associated with another thromboembolic disorder, e.g., a blood clot.

In another aspect, the present invention features a method of reducing the risk of atrial fibrillation, in a subject in need thereof, the method comprising administering to the subject an effective amount of a compound described herein (e.g., Compound 1) or a pharmaceutically acceptable salt thereof, or of a composition described herein (e.g., a composition comprising Compound 1). In some embodiments, the subject is at a high risk of developing atrial fibrillation. In some embodiments, the subject is also in need of dialysis, e.g., renal dialysis. In some embodiments, the compound described herein is administered to the subject while the subject is undergoing dialysis. In some embodiments, the compound or pharmaceutically acceptable salt or composition is administered to the subject before or after receiving dialysis. In some embodiments, the patient has end-stage renal disease. In some embodiments, the subject is not in need of dialysis, e.g., renal dialysis. In some embodiments, the patient is at a high risk for bleeding. In some embodiments, the atrial fibrillation is associated with another thromboembolic disorder, e.g., a blood clot.

In another aspect, the present invention features a method of prophylaxis of atrial fibrillation, in a subject in need thereof, the method comprising administering to the subject an effective amount of a compound described herein (e.g., Compound 1) or a pharmaceutically acceptable salt thereof, or of a composition described herein (e.g., a composition comprising Compound 1). In some embodiments, the subject is at a high risk of developing atrial fibrillation. In some embodiments, the subject is also in need of dialysis, e.g., renal dialysis. In some embodiments, the compound described herein is administered to the subject while the subject is undergoing dialysis. In some embodiments, the compound or pharmaceutically acceptable salt or composition is administered to the subject before or after receiving dialysis. In some embodiments, the patient has end-stage renal disease. In some embodiments, the subject is not in need of dialysis, e.g., renal dialysis. In some embodiments, the patient is at a high risk for bleeding. In some embodiments, the atrial fibrillation is associated with another thromboembolic disorder, e.g., a blood clot.

In another aspect, the present invention features a method of treating heparin-induced thrombocytopenia in a subject in need thereof, the method comprising administering to the subject an effective amount of a compound described herein (e.g., Compound 1) or a pharmaceutically acceptable salt thereof, or of a composition described herein (e.g., a composition comprising Compound 1).

In another aspect, the present invention features a method of reducing the risk of heparin-induced thrombocytopenia in a subject in need thereof, the method comprising administering to the subject an effective amount of a compound described herein (e.g.,

Compound 1) or a pharmaceutically acceptable salt thereof, or of a composition described herein (e.g., a composition comprising Compound 1).

In another aspect, the present invention features a method of prophylaxis of heparin-induced thrombocytopenia in a subject in need thereof, the method comprising administering to the subject an effective amount of a compound described herein (e.g., Compound 1) or a pharmaceutically acceptable salt thereof, or of a composition described herein (e.g., a composition comprising Compound 1).

In another aspect, the present invention features a method of treating heparin-induced thrombocytopenia thrombosis in a subject in need thereof, the method comprising administering to the subject an effective amount of a compound described herein (e.g., Compound 1) or a pharmaceutically acceptable salt thereof, or of a composition described herein (e.g., a composition comprising Compound 1).

In another aspect, the present invention features a method of reducing the risk of heparin-induced thrombocytopenia thrombosis in a subject in need thereof, the method comprising administering to the subject an effective amount of a compound described herein (e.g., Compound 1) or a pharmaceutically acceptable salt thereof, or of a composition described herein (e.g., a composition comprising Compound 1).

In another aspect, the present invention features a method of prophylaxis of heparin-induced thrombocytopenia thrombosis in a subject in need thereof, the method comprising administering to the subject an effective amount of a compound described herein (e.g., Compound 1) or a pharmaceutically acceptable salt thereof, or of a composition described herein (e.g., a composition comprising Compound 1).

In another aspect, the present invention features a method of prophylaxis of a thromboembolic disorder in a subject in need thereof, the method comprising administering to the subject an effective amount of a compound described herein (e.g., Compound 1) or a pharmaceutically acceptable salt thereof, or of a composition described herein (e.g., a composition comprising Compound 1), wherein the subject has cancer or is being with a chemotherapeutic. In some embodiments, the subject is concurrently receiving chemotherapy. In some embodiments, the subject has elevated lactase dehydrogenase levels. In some embodiments, the thromboembolic disorder is venous thromboembolism. In some embodiments, the thromboembolic disorder is deep vein thrombosis. In some embodiments, the thromboembolic disorder is pulmonary embolism.

In another aspect, the present invention features a method of treating thrombotic microangiopathy in a subject in need thereof, the method comprising administering to the subject an effective amount of a compound described herein (e.g., Compound 1) or a pharmaceutically acceptable salt thereof, or of a composition described herein (e.g., a composition comprising Compound 1). In some embodiments, the thrombotic microangiopathy is hemolytic uremic syndrome (HUS). In some embodiments, the thrombotic microangiopathy is thrombotic thrombocytopenic purpura (TTP).

In another aspect, the present invention features a method of reducing the risk of thrombotic microangiopathy in a subject in need thereof, the method comprising administering to the subject an effective amount of a compound described herein (e.g., Compound 1) or a pharmaceutically acceptable salt thereof, or of a composition described herein (e.g., a composition comprising Compound 1). In some embodiments, the thrombotic microangiopathy is hemolytic uremic syndrome (HUS). In some embodiments, the thrombotic microangiopathy is thrombotic thrombocytopenic purpura (TTP).

In another aspect, the present invention features a method of prophylaxis of thrombotic microangiopathy in a subject in need thereof, the method comprising administering to the subject an effective amount of a compound described herein (e.g., Compound 1) or a pharmaceutically acceptable salt thereof, or of a composition described herein (e.g., a composition comprising Compound 1). In some embodiments, the thrombotic microangiopathy is hemolytic uremic syndrome (HUS). In some embodiments, the thrombotic microangiopathy is thrombotic thrombocytopenic purpura (TTP).

In another aspect, the present invention features a method of prophylaxis of recurrent ischemia in a subject in need thereof, the method comprising administering to the subject an effective amount of a compound described herein (e.g., Compound 1) or a pharmaceutically acceptable salt thereof, or of a composition described herein (e.g., a composition comprising Compound 1), wherein the subject has acute coronary syndrome. In some embodiments, the subject has atrial fibrillation. In some embodiments, the subject does not have atrial fibrillation. In another aspect, the present invention features a method of treating a subject identified as being at risk, e.g., high risk, for stroke (e.g., large vessel acute ischemic stroke) or thrombosis thereby reducing the likelihood of stroke or thrombosis in the subject. In some embodiments, the subject is further identified as being at risk for bleeding (e.g., excessive bleeding) or sepsis. In some embodiments, the treatment is effective without bleeding liabilities. In some embodiments, the treatment is effective to maintain the patency of infusion ports and lines. In addition, the compounds described herein (e.g., Compound 1) are useful in the treatment and prevention of other diseases in which the generation of thrombin has been implicated as playing a physiologic role. For example, thrombin has been implicated in contributing to the morbidity and mortality of chronic and degenerative diseases, such as cancer, arthritis, atherosclerosis, vascular dementia, and Alzheimer's disease, by its ability to regulate many different cell types through specific cleavage and activation of a cell surface thrombin receptor, mitogenic effects, diverse cellular functions such as cell proliferation, for example, abnormal proliferation of vascular cells resulting in restenosis or angiogenesis, release of PDGF, and DNA synthesis. Inhibition of Factor XIa effectively blocks thrombin generation and therefore neutralizes any physiologic effects of thrombin on various cell types. The representative indications discussed above include some, but not all, of the potential clinical situations amenable to treatment with a Factor XIa inhibitor.

In another aspect, the present invention features a method of treating a subject that has edema (e.g., angioedema, e.g., hereditary angioedema), comprising administering Compound 1 or a pharmaceutically acceptable salt thereof, or a composition described herein (e.g., a composition comprising Compound 1) to the subject.

In another aspect, the present invention features a method of prophylaxis of edema (e.g., angioedema, e.g., hereditary angioedema) in a subject, comprising administering Compound 1 or a pharmaceutically acceptable salt thereof, or a composition described herein (e.g., a composition comprising Compound 1) to the subject.

In another aspect, the present invention features a method of reducing the risk of edema (e.g., angioedema, e.g., hereditary angioedema) in a subject, comprising administering Compound 1 or a pharmaceutically acceptable salt thereof, or a composition described herein (e.g., a composition comprising Compound 1) to the subject.

In another aspect, the present invention features a method of inhibiting kallikrein in a subject, comprising administering to the subject with edema (e.g., angioedema, e.g., hereditary angioedema), an effective amount of Compound 1 or a pharmaceutically acceptable salt thereof, or of a composition described herein (e.g., a composition comprising Compound 1) to the subject.

In another aspect, the present invention features a method of treating a thromboembolic consequence or complication in a subject, comprising administering to a subject an effective amount of Compound 1 or a pharmaceutically acceptable salt thereof, or of a composition described herein (e.g., a composition comprising Compound 1). In some embodiments, the thromboembolic consequence or complication is associated with a peripheral vascular intervention (e.g., of the limbs), hemodialysis, catheter ablation, a cerebrovascular intervention, transplantation of an organ (e.g., liver), surgery (e.g., orthopedic surgery, lung surgery, abdominal surgery, or cardiac surgery, (e.g., open-heart surgery)), a trans-catheter aortic valve implantation, a large bore intervention used to treat an aneurysm, a percutaneous coronary intervention, or hemophilia therapy. In some embodiments, the surgery is orthopedic surgery, lung surgery, abdominal surgery, or cardiac surgery. In some embodiments, the cardiac surgery is complex cardiac surgery or lower risk cardiac surgery. In some embodiments, the thromboembolic consequence or complication is associated with a percutaneous coronary intervention.

In another aspect, the present invention features a method of prophylaxis of a thromboembolic consequence or complication in a subject, comprising administering to a subject an effective amount of Compound 1 or a pharmaceutically acceptable salt thereof, or of a composition described herein (e.g., a composition comprising Compound 1). In some embodiments, the thromboembolic consequence or complication is associated with a peripheral vascular intervention (e.g., of the limbs), hemodialysis, catheter ablation, e.g., catheter ablation for atrial fibrillation, a cerebrovascular intervention, transplantation of an organ (e.g., liver), surgery (e.g., orthopedic surgery, lung surgery, abdominal surgery, or cardiac surgery, (e.g., open-heart surgery)), a trans-catheter aortic valve implantation, a large bore intervention used to treat an aneurysm, a percutaneous coronary intervention, or hemophilia therapy. In some embodiments, the surgery is orthopedic surgery, lung surgery, abdominal surgery, or cardiac surgery. In some embodiments, the cardiac surgery is complex cardiac surgery or lower risk cardiac surgery. In some embodiments, the thromboembolic consequence or complication is associated with a percutaneous coronary intervention.

In another aspect, the present invention features a method of reducing the risk of a thromboembolic consequence or complication in a subject, comprising administering to a subject an effective amount of Compound 1 or a pharmaceutically acceptable salt thereof, or of a composition described herein (e.g., a composition comprising Compound 1). In some embodiments, the thromboembolic consequence or complication is associated with a peripheral vascular intervention (e.g., of the limbs), hemodialysis, catheter ablation, e.g., catheter ablation for atrial fibrillation, a cerebrovascular intervention, transplantation of an organ (e.g., liver), surgery (e.g., orthopedic surgery, lung surgery, abdominal surgery, or cardiac surgery, (e.g., open-heart surgery)), a trans-catheter aortic valve implantation, a large bore intervention used to treat an aneurysm, a percutaneous coronary intervention, or hemophilia therapy. In some embodiments, the surgery is orthopedic surgery, lung surgery, abdominal surgery, or cardiac surgery. In some embodiments, the cardiac surgery is complex cardiac surgery or lower risk cardiac surgery. In some embodiments, the thromboembolic consequence or complication is associated with a percutaneous coronary intervention.

In another aspect, the invention features a method of treating restenosis following arterial injury in a subject, comprising administering to a subject an effective amount of Compound 1 or a pharmaceutically acceptable salt thereof, or of a composition described herein (e.g., a composition comprising Compound 1). In some embodiments, the arterial injury occurs after a cranial artery stenting.

In another aspect, the present invention features a method of prophylaxis of restenosis following arterial injury in a subject, comprising administering to a subject an effective amount of Compound 1 or a pharmaceutically acceptable salt thereof, or of a composition described herein (e.g., a composition comprising Compound 1). In some embodiments, the arterial injury occurs after a cranial artery stenting.

In another aspect, the present invention features a method of reducing the risk of restenosis following arterial injury in a subject, comprising administering to a subject an effective amount of Compound 1 or a pharmaceutically acceptable salt thereof, or of a composition described herein (e.g., a composition comprising Compound 1). In some embodiments, the arterial injury occurs after a cranial artery stenting.

In another aspect, the present invention features a method of treating hepatic vessel thrombosis in a subject, comprising administering to a subject an effective amount of Compound 1 or a pharmaceutically acceptable salt thereof, or of a composition described herein (e.g., a composition comprising Compound 1).

In another aspect, the present invention features a method of prophylaxis of hepatic vessel thrombosis in a subject, comprising administering to a subject an effective amount of Compound 1 or a pharmaceutically acceptable salt thereof, or of a composition described herein (e.g., a composition comprising Compound 1).

In another aspect, the present invention features a method of reducing the risk of hepatic vessel thrombosis in a subject, comprising administering to a subject an effective amount of Compound 1 or a pharmaceutically acceptable salt thereof, or of a composition described herein (e.g., a composition comprising Compound 1).

In another aspect, the present invention features a method of treating a non-ST-elevation myocardial infarction or ST-elevation myocardial infarction), comprising administering to a subject an effective amount of Compound 1 or a pharmaceutically acceptable salt thereof, or of a composition described herein (e.g., a composition comprising Compound 1).

In another aspect, the present invention features a method of prophylaxis of a non-ST-elevation myocardial infarction or ST-elevation myocardial infarction in a subject, comprising administering to the subject an effective amount of Compound 1 or a pharmaceutically acceptable salt thereof, or of a composition described herein (e.g., a composition comprising Compound 1).

In another aspect, the present invention features a method of reducing the risk of a non-ST-elevation myocardial infarction or ST-elevation myocardial infarction in a subject, comprising administering to the subject an effective amount of Compound 1 or a pharmaceutically acceptable salt thereof, or of a composition described herein (e.g., a composition comprising Compound 1).

In another aspect, the present invention features a method of maintaining blood vessel patency, comprising administering to a subject an effective amount of Compound 1 or a pharmaceutically acceptable salt thereof, or of a composition described herein (e.g., a composition comprising Compound 1). In some embodiments, the subject has acute kidney injury. In some embodiments, the subject additionally undergoes continuous renal replacement therapy.

In some embodiments of any of the foregoing, the compound described herein or composition thereof is administered orally or parenterally. In certain embodiments, the compound or composition thereof is administered orally. In certain embodiments, the compound or composition thereof is administered after the subject has discontinued use of a direct oral anticoagulant. In certain embodiments, the subject used the direct oral anticoagulant for up to about 2.5 years. In certain embodiments, the subject is a mammal, e.g., a human.

In some embodiments of the methods described herein, the pharmaceutically acceptable salt of the compound is a hydrochloride salt. In some embodiments, the compound is administered to the subject intravenously. In some embodiments, the compound is administered to the subject subcutaneously. In some embodiments, the compound is administered to the subject as a continuous intravenous infusion. In some embodiments, the compound is administered to the subject as a bolus. In some embodiments, the subject is a human. In some embodiments, the subject has an elevated risk of a thromboembolic disorder. In some embodiments, the thromboembolic disorder is a result of a complication in surgery.

In some embodiments, the subject is sensitive to or has developed sensitivity to heparin. In some embodiments, the subject is resistant to or has developed resistance to heparin.

In some embodiments, the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

### BRIEF DESCRIPTION OF THE DRAWINGS

FIG. 1 depicts an exemplary XRPD pattern of Compound 1.HCl (Pattern A).

FIG. 2 depicts an exemplary optical microscopic image of Compound 1.HCl (Pattern A).

FIG. 3 depicts an exemplary DVS kinetic graph of Compound 1.HCl (Pattern A).

FIG. 4 depicts an exemplary DVS isotherm of Compound 1.HCl (Pattern A).

FIG. 5 depicts exemplary XRPD patterns of Compound 1.HCl before and after DVS.

FIG. 6 depicts an exemplary DSC thermogram of Compound 1.HCl (Pattern A).

FIG. 7 depicts an exemplary 1H-NMR of Compound 1.HCl (Pattern A).

FIG. 8 depicts an exemplary TGA of Compound 1.HCl (Pattern A).

FIG. 9 depicts an exemplary comparison of XRPD patterns of Samples 16 and 18.

FIG. 10 depicts an exemplary comparison of XRPD patterns of Samples 16 and 18 with Compound 1.HCl, Pattern A.

FIG. 11 depicts an exemplary optical microscopic image of amorphous Compound 1.HCl (Sample 16).

FIG. 12 depicts an exemplary optical microscopic image of semi-crystalline Compound 1.HCl (Sample 18).

FIG. 13 depicts an exemplary 1H-NMR of amorphous form (Sample 16).

FIG. 14 depicts an exemplary DSC thermogram of amorphous form (Sample 16).

FIG. 15 depicts an exemplary overlay of DSC thermogram and the TGA of Sample C1 (Compound 1.HCl).

FIG. 16 depicts an exemplary 1H-NMR of Sample C1 (Compound 1.HCl).

FIG. 17 depicts an exemplary DVS isotherm of amorphous Compound 1.HCl (Sample C1).

FIG. 18 depicts an exemplary Comparison of XRPD patterns of pre and post DVS Sample C1 with Compound 1.HCl, Pattern A.

FIG. 19 depicts an exemplary optical microscopic image of amorphous Compound 1.HCl (Sample C1) before (left) and after (right) DVS experiment.

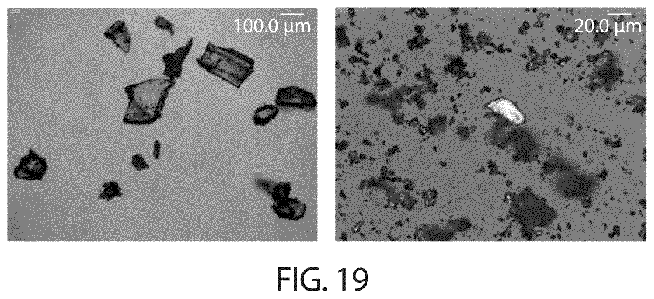

FIG. 20 depicts an exemplary comparison of XRPD patterns Pattern A with Sample D9 (after heating amorphous salt at 140° C.).

FIG. 21 depicts an exemplary DSC overlay of amorphous Compound 1.HCl (Sample C1) thermogram with Sample D9 (after heating amorphous salt at 140° C.).

FIG. 22 depicts an exemplary XRPD comparison of neat and solvent drop grinding with Pattern A.

FIG. 23 depicts an exemplary XRPD comparison of vapor diffusion experiments of amorphous Compound 1.HCl with Pattern A.

FIG. 24 depicts an exemplary XRPD comparison of competitive slurries in different solvents at T=5 minutes with Pattern A.

FIG. 25 depicts an exemplary XRPD comparison of competitive slurries in different solvents at T=24 h with Pattern A.

FIG. 26 depicts an exemplary XRPD pattern of Compound 1.HCl, Pattern A.

FIG. 27 depicts the pressure gradient across membrane oxygenator for cardiopulmonary bypass experiment conducted in the hound model.

FIG. 28 depicts a comparison of plasma concentrations and activated partial thromboplastin time (aPTT) ratio measured in the hound model.

FIG. 29 depicts the activated partial thromboplastin time (aPTT) measured in the hound model following Compound 1 administration.

### DETAILED DESCRIPTION

### Definitions

As used herein, “XRPD” refers to X-ray powder diffraction. As used herein, “TGA” refers to thermogravimetric analysis. As used herein, “DSC” refers to differential scanning calorimetry. As used herein, “NMR” refers to nuclear magnetic resonance. As used herein, “DVS” refers to dynamic vapor sorption. As used herein, “EtOAc” refers to ethyl acetate. As used herein, “MeOH” refers to methanol. As used herein, “EtOH” refers to ethanol. As used herein, “RH” refers to relative humidity.

As used herein, “crystalline” refers to a solid having a highly regular chemical structure, i.e., having long range structural order in the crystal lattice. The molecules are arranged in a regular, periodic manner in the 3-dimensional space of the lattice. In particular, a crystalline form may be produced as one or more single crystalline forms. For the purposes of this application, the terms “crystalline form”, “single crystalline form,” “crystalline solid form,” “solid form,” and “polymorph” are synonymous and used interchangeably; the terms distinguish between crystals that have different properties (e.g., different XRPD patterns and/or different DSC scan results).

The term “substantially crystalline” refers to forms that may be at least a particular weight percent crystalline. Particular weight percentages are 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, Compound 1 can be a substantially crystalline sample of any of the crystalline solid forms described herein.

The term “substantially pure” relates to the composition of a specific crystalline solid form of Compound 1 that may be at least a particular weight percent free of impurities and/or other solid forms of Compound 1 or a pharmaceutically acceptable salt thereof. Particular weight percentages are 70%, 75%, 80%, 85%, 90%, 95%, 99%, or any percentage between 70% and 100%. In some embodiments, a crystalline solid form of Compound 1 or a pharmaceutically acceptable salt thereof as described herein is substantially pure at a weight percent between 95% and 100%, e.g., about 95%, about 96%, about 97%, about 98%, about 99%, or about 99.9%.

As used herein, the term “anhydrous” or “anhydrate” when referring to a crystalline form of Compound 1 means that no solvent molecules, including those of water, form a portion of the unit cell of the crystalline form. A sample of an anhydrous crystalline form may nonetheless contain solvent molecules that do not form part of the unit cell of the anhydrous crystalline form, e.g., as residual solvent molecule left behind from the production of the crystalline form. In a preferred embodiment, a solvent can make up 0.5% by weight of the total composition of a sample of an anhydrous form. In a more preferred embodiment, a solvent can make up 0.2% by weight of the total composition of a sample of an anhydrous form. In some embodiments, a sample of an anhydrous crystalline form of Compound 1 contains no solvent molecules, e.g., no detectable amount of solvent. The term “solvate” when referring to a crystalline form of Compound 1 means that solvent molecules, e.g., organic solvents and water, form a portion of the unit cell of the crystalline form. Solvates that contain water as the solvent are also referred to herein as “hydrates.” The term “isomorphic” when referring to a crystalline form of Compound 1 means that the form can comprise different chemical constituents, e.g., contain different solvent molecules in the unit cell, but have identical XRPD patterns. Isomorphic crystalline forms are sometimes referred to herein as “isomorphs.”

A crystalline form of Compound 1 described herein can melt at a specific temperature or across a range of temperatures. Such a specific temperature or range of temperatures can be represented by the onset temperature (Tₒₙₛₑₜ) of the melting endotherm in the crystalline form's DSC trace. In some embodiments, at such an onset temperature, a sample of a crystalline form of Compound 1 melts and undergoes a concurrently occurring side-process, e.g., recrystallization or chemical decomposition. In some embodiments, at such an onset temperature, a crystalline form of Compound 1 melts in the absence of other concurrently occurring processes.

The term “characteristic peaks” when referring to the peaks in an XRPD pattern of a crystalline form of Compound 1 refers to a collection of certain peaks whose values of 2θ across a range of 0°-40° are, as a whole, uniquely assigned to one of the crystalline forms of Compound 1.

As used herein, “slurrying” refers to a method wherein a compound as described herein is suspended in a solvent (e.g., polar aprotic solvent or nonpolar solvent) and is collected again (e.g., by filtration) after agitating the suspension.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also, “therapeutic treatment”).

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

Disease, disorder, and condition are used interchangeably herein.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. In some embodiments, the pediatric subject is between the age of 0 and 18 years old. In some embodiments, the adult subject is beyond 18 years old.

As used herein, the term “artificial surface” refers to any non-human or non-animal surface that comes into contact with blood of the subject, for example, during a medical procedure. It can be a vessel for collecting or circulating blood of a subject outside the subject's body. It can also be a stent, valve, intraluminal catheter or a system for pumping blood. By way of non-limiting example such artificial surfaces can be steel, any type of plastic, glass, silicone, rubber, etc. In some embodiments, the artificial surface is exposed to at least 50%. 60%, 70% 80%, 90% or 100% of the blood of subject.

As used herein, the term “conditioning” or “conditioned” with respect to an artificial surface refers to priming or flushing the artificial surface (e.g., extracorporeal surface) with a compound described herein (e.g., Compound 1) or a pharmaceutically acceptable salt thereof, already in a priming or flushing solution (e.g., blood, a saline solution, Ringer's solution) or as a separate administration to the artificial surface prior to, during, or after a medical procedure.

Compounds

Described herein are compounds that inhibit Factor XIa or kallikrein.

In one aspect, provided herein is a crystalline pharmaceutically acceptable salt of Formula (I):

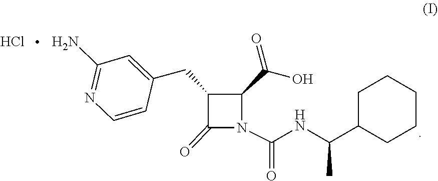 [SMILES: Cl ; C[C@@H](NC(=O)N1C(=O)[C@H](Cc2ccnc(N)c2)[C@H]1C(=O)O)C1CCCCC1]

The crystalline pharmaceutically acceptable salt of Formula (I) is a hydrochloride salt of Compound 1 and also referred to herein as Compound 1.HCl.

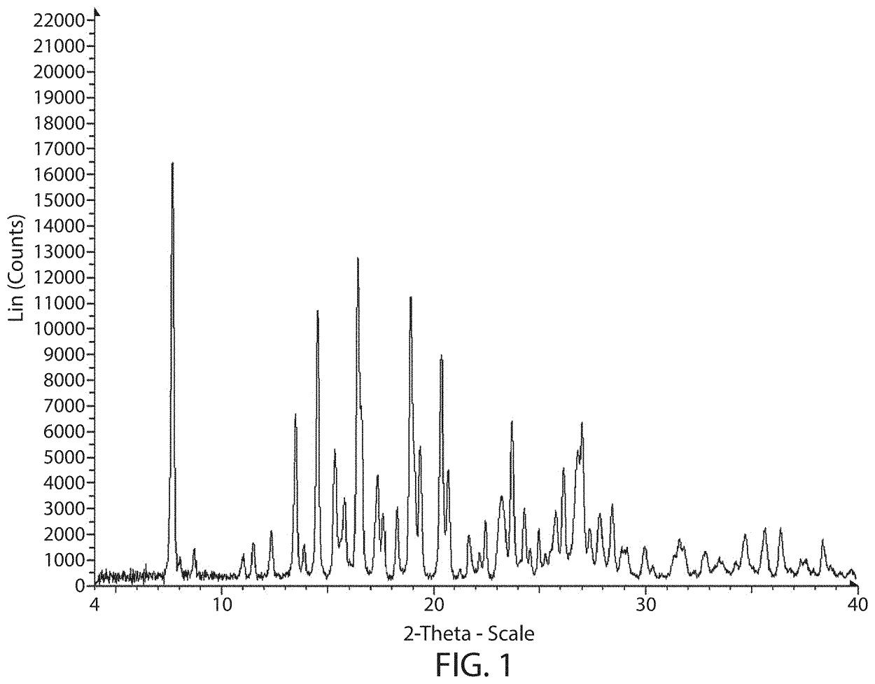

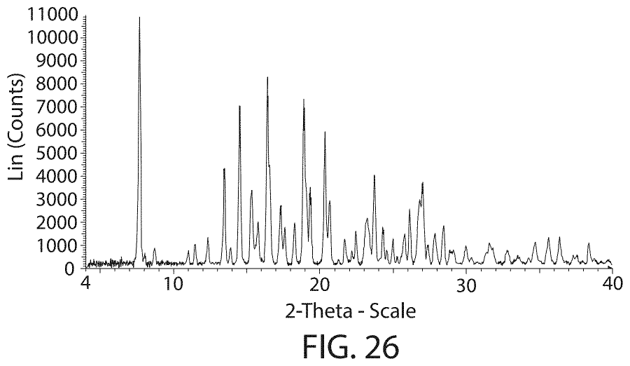

In some embodiments, the crystalline pharmaceutically acceptable salt of Formula (I) has an XRPD pattern with characteristic peaks between and including the following values of 2θ in degrees: 7.4 to 7.8, 13.3 to 13.7, 14.3 to 14.7, 15.2 to 15.6, 16.3 to 16.7, 17.2 to 17.6, 18.8 to 19.2, 20.2 to 20.6, 23.5 to 23.9, and 26.7 to 27.1. In some embodiments, the crystalline pharmaceutically acceptable salt of Formula (I) has an XRPD pattern with characteristic peaks at the following values of 2θ in degrees: 7.6, 13.5, 14.5, 15.4, 16.5, 17.4, 19.0, 20.4, 23.7, and 26.9. In some embodiments, the crystalline pharmaceutically acceptable salt of Formula (I) has an XRPD pattern with characteristic peaks between and including the following values of 2θ in degrees: 7.4 to 7.8, 14.3 to 14.7, 16.3 to 16.7, 18.8 to 19.2, and 20.2 to 20.6. In some embodiments, the crystalline pharmaceutically acceptable salt of Formula (I) has an XRPD pattern with characteristic peaks at the following values of 2θ in degrees: 7.6, 14.5, 16.5, 19.0, and 20.4. In some embodiments, the crystalline pharmaceutically acceptable salt of Formula (I) has an XRPD pattern substantially as depicted in FIG. 1. In some embodiments, the crystalline pharmaceutically acceptable salt of Formula (I) has an XRPD pattern substantially as depicted in FIG. 26.

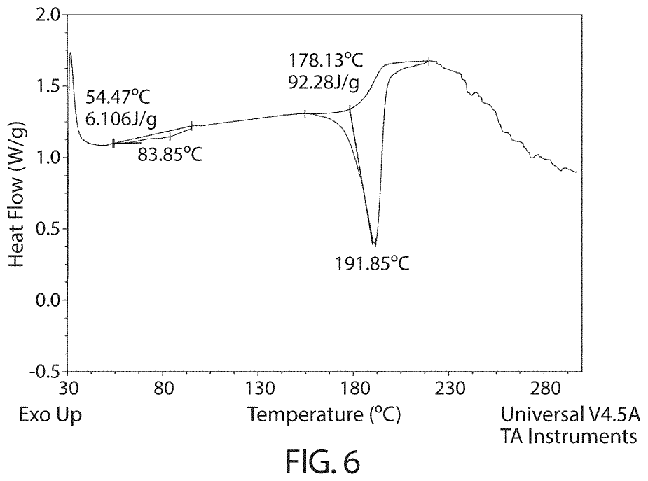

In some embodiments, the crystalline pharmaceutically acceptable salt of Formula (I) melts at a Tₒₙₛₑₜ from about 178° C. to about 192° C. as determined by DSC at a ramp rate of 10° C./min. In some embodiments, the crystalline pharmaceutically acceptable salt of Formula (I) has a DSC thermogram substantially as depicted in FIG. 6.

In an aspect, described herein is an amorphous pharmaceutically acceptable salt of Formula (I)

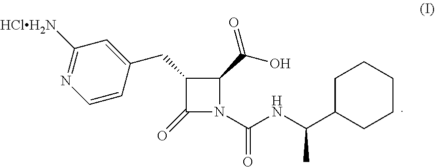

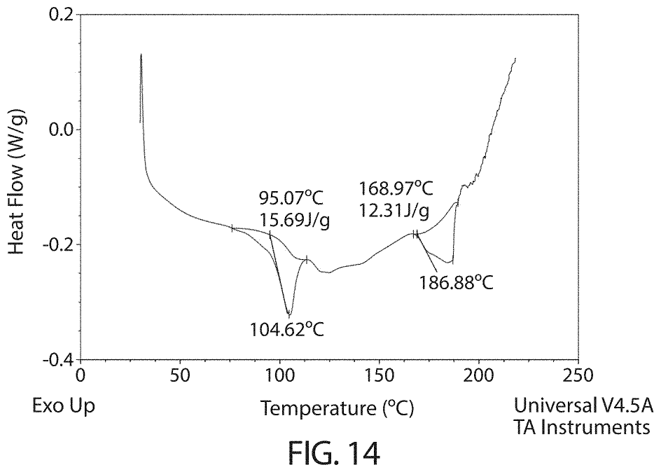

In some embodiments, the amorphous pharmaceutically acceptable salt of Formula (I) has an endotherm at a Tₒₙₛₑₜ from about 95° C. to about 105° C. as determined by DSC at a ramp rate of 10° C./min. In some embodiments, the amorphous pharmaceutically acceptable salt of Formula (I), has a DSC thermogram substantially as depicted in FIG. 14. In some embodiments, the amorphous pharmaceutically acceptable salt of Formula (I), when subjected to a temperature of about 140° C., transforms into the crystalline compound of Formula (I) as indicated by DSC at a ramp rate of 10° C./min.

In some embodiments, a compound described herein is formed into a salt. A compound described herein can be administered as a free acid, a zwitterion or as a salt. A salt can also be formed between a cation and a negatively charged substituent on a compound described herein. Suitable cationic counterions include sodium ions, potassium ions, magnesium ions, calcium ion, and ammonium ions (e.g., a tetraalkyl ammonium cation such as tetramethylammonium ion). In compounds including a positively charged substituent or a basic substituent, a salt can be formed between an anion and a positively charged substituent (e.g., amino group) or basic substituent (e.g., pyridyl) on a compound described herein.

Suitable anions include chloride, bromide, iodide, sulfate, nitrate, phosphate, citrate, methanesulfonate, trifluoroacetate, and acetate.

Pharmaceutically acceptable salts of the compounds described herein (e.g., a pharmaceutically acceptable salt of Compound 1) also include those derived from pharmaceutically acceptable inorganic and organic acids and bases. Examples of suitable acid salts include acetate, 4-acetamidobenzoate, adipate, alginate, 4-aminosalicylate, aspartate, ascorbate, benzoate, benzenesulfonate, bisulfate, butyrate, citrate, camphorate, camphorsulfonate, carbonate, cinnamate, cyclamate, decanoate, decanedioate, 2,2-dichloroacetate, digluconate, dodecylsulfate, ethanesulfonate, ethane-1,2-disulfonate, formate, fumarate, galactarate, glucoheptanoate, gluconate, glucoheptonate, glucoronate, glutamate, glutarate, glycerophosphate, glycolate, hemisulfate, heptanoate, hexanoate, hippurate, hydrochloride, hydrobromide, hydroiodide, 1-hydroxy-2-naphthoate, 2-hydroxyethanesulfonate, isobutyrate, lactate, lactobionate, laurate, malate, maleate, malonate, mandelate, methanesulfonate, naphthalene-1,5-disulfonate, 2-naphthalenesulfonate, nicotinate, nitrate, octanoate, oleate, oxalate, 2-oxoglutarate, palmitate, palmoate, pectinate, 3-phenylpropionate, phosphate, phosphonate, picrate, pivalate, propionate, pyroglutamate, salicylate, sebacate, succinate, stearate, sulfate, tartrate, thiocyanate, toluenesulfonate, tosylate, and undecanoate.

Salts derived from appropriate bases include alkali metal (e.g., sodium), alkaline earth metal (e.g., magnesium), ammonium and (alkyl)4N+salts. This invention also envisions the quaternization of any basic nitrogen-containing groups of the compounds disclosed herein. Water or oil-soluble or dispersible products may be obtained by such quaternization.

As used herein, the compounds of this invention, including the Compound 1, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood), or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

Any formula or a compound described herein is also intended to represent unlabeled forms as well as isotopically labeled forms of the compounds, isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as 2H, 3H, 11C, 13C, 14C, 15N, 18F 51P, 32P, 35S, 36Cl, 125I respectively. The invention includes various isotopically labeled compounds as defined herein, for example, those into which radioactive isotopes, such as 3H, 13C, and 14C are present. Such isotopically labelled compounds are useful in metabolic studies (with 14C), reaction kinetic studies (with, for example ′H or 3H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies, isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of a formula described herein. The concentration of such a heavier isotope, specifically deuterium, may be defined by the isotopic enrichment factor. The term “isotopic enrichment factor” as used herein means the ratio between the isotopic abundance and the natural abundance of a specified isotope If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 8633.3 (99.5% deuterium incorporation).

Isotopically-labelled compounds described herein can generally be prepared by conventional techniques known to those skilled in the art or by processes analogous to those described in the accompanying Examples and Preparations using an appropriate isotopically-labeled reagents in place of the non-labeled reagent previously employed. Pharmaceutically acceptable solvates in accordance with the invention include those wherein the solvent of crystallization may be isotopically substituted, e.g, D2O, D6-acetone, D6-DMSO.

Any asymmetric atom (e.g., carbon or the like) of the compound(s) of the present invention can be present in racemic or enantiomerically enriched, for example the (R)- (S)- or (RS)-configuration, in certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof. Any resulting mixtures of isomers can be separated on the basis of the physicochemical differences of the constituents, into the pure or substantially pure geometric or optical isomers, diastereomers, racemates, for example, by chromatography or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. An acidic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, (+)-O,O′-Di-p-toluoyl-D-tartaric acid, mandelic acid, malic acid or camphor-10-sulfonic acid. Racemic products can also be resolved by chiral chromatography, e.g., high pressure liquid chromatography (HPLC) using a chiral adsorbent.

The compounds described herein (e.g., Compound 1) may also be represented in multiple tautomeric forms. In such instances, the invention expressly includes all tautomeric forms of the compounds described herein. All crystal forms of the compounds described herein are expressly included in this invention.

A compound described herein (e.g., Compound 1) can be evaluated for its ability to modulate (e.g., inhibit) Factor XIa or kallikrein.

### Methods of Treatment, Prophylaxis, or Reduction of Risk

The compounds described herein (e.g., Compound 1) can inhibit Factor XIa or kallikrein. In some embodiments, a compound described herein can inhibit both Factor XIa and kallikrein. As a result, these compounds can be useful in the treatment, prophylaxis, or reduction in the risk of a disorder described herein. Exemplary disorders include thrombotic events associated with coronary artery and cerebrovascular disease, venous or arterial thrombosis, coagulation syndromes, ischemia (e.g., coronary ischemia) and angina (stable and unstable), deep vein thrombosis (DVT), hepatic vein thrombosis, disseminated intravascular coagulopathy, Kasabach-Merritt syndrome, pulmonary embolism, myocardial infarction (e.g., ST-elevation myocardial infarction or non-ST-elevation myocardial infarction (e.g., non-ST-elevation myocardial infarction before catheterization), cerebral infarction, cerebral thrombosis, transient ischemic attacks, atrial fibrillation (e.g., non-valvular atrial fibrillation), cerebral embolism, thromboembolic complications of surgery (e.g., hip or knee replacement, orthopedic surgery, cardiac surgery, lung surgery, abdominal surgery, or endarterectomy) and peripheral arterial occlusion and may also be useful in treating or preventing myocardial infarction, stroke (e.g., large vessel acute ischemic stroke), angina and other consequences of atherosclerotic plaque rupture. The compounds of the invention possessing Factor XIa or kallikrein inhibition activity may also be useful in preventing thromboembolic disorders, e.g., venous thromboembolisms, in cancer patients, including those receiving chemotherapy and/or those with elevated lactase dehydrogenase (LDH) levels, and to prevent thromboembolic events at or following tissue plasminogen activator-based or mechanical restoration of blood vessel patency. The compounds of the invention possessing Factor XIa or kallikrein inhibition activity may also be useful as inhibitors of blood coagulation such as during the preparation, storage and fractionation of whole blood. Additionally, the compounds described herein may be used in acute hospital settings or periprocedurally, where a patient is at risk of a thromboembolic disorder or complication, and also in patients who are in a heightened coagulation state, e.g., cancer patients.

Factor XIa inhibition, according to the present invention, can be a more effective and safer method of inhibiting thrombosis compared to inhibiting other coagulation serine proteases such as thrombin or Factor Xa. Administration of a small molecule Factor XIa inhibitor should have the effect of inhibiting thrombin generation and clot formation with no or substantially no effect on bleeding times and little or no impairment of haemostasis. These results differ substantially from that of other “direct acting” coagulation protease inhibitors (e.g., active-site inhibitors of thrombin and Factor Xa), which demonstrate prolongation of bleeding time and less separation between antithrombotic efficacy and bleeding time prolongation. A preferred method according to the invention comprises administering to a mammal a pharmaceutical composition containing at least one compound of the invention.

The compounds described herein (e.g., Compound 1) can inhibit kallikrein. As a result, these compounds can be useful in the treatment, prophylaxis, or reduction in the risk of diseases involved in inflammation, such as edema (e.g., cerebral edema, macular edema, and angioedema (e.g., hereditary angioedema)). In some embodiments, the compounds of the invention can be useful in the treatment or prevention of hereditary angioedema. The compounds described herein (e.g., Compound 1) can also be useful in the treatment, prophylaxis, or reduction in the risk of, e.g., stroke, ischemia (e.g., coronary ischemia), and perioperative blood loss for example, Compound 1. The methods of the present invention are useful for treating or preventing those conditions which involve the action of Factor XIa or kallikrein. Accordingly, the methods of the present invention are useful in treating consequences of atherosclerotic plaque rupture including cardiovascular diseases associated with the activation of the coagulation cascade in thrombotic or thrombophilic states.

More particularly, the methods of the present invention can be used in the treatment, prophylaxis, or reduction in the risk of acute coronary syndromes such as coronary artery disease, myocardial infarction, unstable angina (including crescendo angina), ischemia (e.g., ischemia resulting from vascular occlusion), and cerebral infarction. The methods of the present invention further may be useful in the treatment, prophylaxis, or reduction in the risk of stroke (e.g., large vessel acute ischemic stroke) and related cerebral vascular diseases (including cerebrovascular accident, vascular dementia, and transient ischemic attack); venous thrombosis and thrombo-embolism, such as deep vein thrombosis (DVT) and pulmonary embolism; thrombosis associated with atrial fibrillation, ventricular enlargement, dilated cardiac myopathy, or heart failure; peripheral arterial disease and intermittent claudication; the formation of atherosclerotic plaques and transplant atherosclerosis; restenosis following arterial injury induced endogenously (by rupture of an atherosclerotic plaque), or exogenously (by invasive cardiological procedures such as vessel wall injury resulting from angioplasty or post-cranial artery stenting); disseminated intravascular coagulopathy, Kasabach-Merritt syndrome, cerebral thrombosis, and cerebral embolism.

Additionally, the methods of the present invention can be used in the treatment, prophylaxis (e.g., preventing), or reduction in the risk of thromboembolic consequences or complications associated with cancer, thrombectomy, surgery (e.g., hip replacement, orthopedic surgery), endarterectomy, introduction of artificial heart valves, peripheral vascular interventions (e.g., of the limbs), cerebrovascular interventions, large bore interventions used in the treatment of aneurysms, vascular grafts, mechanical organs, and implantation (e.g., trans-catheter aortic valve implantation) or transplantation of organs, (e.g., transplantation of the liver), tissue, or cells); percutaneous coronary interventions; catheter ablation; hemophilia therapy; hemodialysis; medications (such as tissue plasminogen activator or similar agents and surgical restoration of blood vessel patency) in patients suffering myocardial infarction, stroke (e.g., large vessel acute ischemic stroke), pulmonary embolism and like conditions; medications (such as oral contraceptives, hormone replacement, and heparin, e.g., for treating heparin-induced thrombocytopenia); sepsis (such as sepsis related to disseminated intravascular coagulation); pregnancy or childbirth; and another chronic medical condition. The methods of the present invention may be used to treat thrombosis due to confinement (e.g., immobilization, hospitalization, bed rest, or limb immobilization, e.g., with immobilizing casts, etc.). In some embodiments, the thromboembolic consequence or complication is associated with a percutaneous coronary intervention.

Additionally, the compounds described herein (e.g., Compound 1) or pharmaceutically acceptable salts thereof or compositions thereof can be useful in the treatment, prophylaxis and reduction in the risk of a thromboembolic disorder, e.g., a venous thromboembolism, deep vein thrombosis or pulmonary embolism, or associated complication in a subject, wherein the subject is exposed to an artificial surface. The artificial surface can contact the subject's blood, for example, as an extracorporeal surface or that of an implantable device. Such artificial surfaces include, but are not limited to, those of dialysis catheters, cardiopulmonary bypass circuits, artificial heart valves, e.g., mechanical heart valves (MHVs), ventricular assist devices, small caliber grafts, central venous catheters, extracorporeal membrane oxygenation (ECMO) apparatuses. Further, the thromboembolic disorder or associated complication may be caused by the artificial surface or associated with the artificial surface. For example, foreign surfaces and various components of mechanical heart valves (MI-IVs) are pro-thrombotic and promote thrombin generation via the intrinsic pathway of coagulation. Further, thrombin and FXa inhibitors are contraindicated with thromboembolic disorders or associated complications caused by artificial surfaces such as those MHVs, as these inhibitors are ineffective at blocking the intrinsic pathway at plasma levels that will not cause heavy bleeding. The compounds of the present invention, which can be used as, for example, Factor XIa inhibitors, are thus contemplated as alternative therapeutics for these purposes.

The compounds described herein (e.g., Compound 1) or pharmaceutically acceptable salts thereof or compositions thereof can also be useful for the treatment, prophylaxis, or reduction in the risk of atrial fibrillation in a subject in need thereof. For example, the subject can have a high risk of developing atrial fibrillation. The subject can also in need of dialysis, such as renal dialysis. The compounds described herein (e.g., Compound 1) or pharmaceutically acceptable salts thereof or compositions thereof can be administered before, during, or after dialysis. Direct oral anticoagulants (DOACs) currently available on the market, such as certain FXa or thrombin inhibitors, are contraindicated for atrial fibrillation under such a condition. The compounds of the present invention, which can be used as, for example, Factor XIa inhibitors, are thus contemplated as alternative therapeutics for these purposes. Additionally, the subject can be at a high risk of bleeding.

In some embodiments, the subject can have end-stage renal disease. In other cases, the subject is not in need of dialysis, such as renal dialysis. Further, the atrial fibrillation can be associated with another thromboembolic disorder such as a blood clot.

Furthermore, the compounds described herein (e.g., Compound 1) or pharmaceutically acceptable salts thereof or compositions thereof can be used in the treatment, prophylaxis, or reduction in the risk of hypertension, e.g., arterial hypertension, in a subject. In some embodiments, the hypertension, e.g., arterial hypertension, can result in atherosclerosis. In some embodiments, the hypertension can be pulmonary arterial hypertension.

Furthermore, the compounds described herein (e.g., Compound 1) or pharmaceutically acceptable salts thereof or compositions thereof can be used in the treatment, prophylaxis, or reduction in the risk of disorders such as heparin-induced thrombocytopenia, heparin-induced thrombocytopenia thrombosis, or thrombotic microangiopathy, e.g., hemolytic uremic syndrome (HUS) or thrombotic thrombocytopenic purpura (TTP).

In some embodiments, the subject is sensitive to or has developed sensitivity to heparin. Heparin-induced thrombocytopenia (HIT) is the development of (a low platelet count), due to the administration of various forms of heparin. HIT is caused by the formation of abnormal antibodies that activate platelets. HIT can be confirmed with specific blood tests. In some embodiments, the subject is resistant to or has developed resistance to heparin. For example, activated clotting time (ACT) test can be performed on the subject to test for sensitivity or resistance towards heparin. The ACT test is a measure of the intrinsic pathway of coagulation that detects the presence of fibrin formation. A subject who is sensitive and/or resistant to standard dose of heparin typically do not reach target anticoagulation time. Common correlates of heparin resistance include, but are not limited to, previous heparin and/or nitroglycerin drips and decreased antithrombin III levels. In some embodiments, the subject has previously been administered an anticoagulant (e.g. bivalirudin/Angiomax).

The compounds described herein (e.g., Compound 1) or pharmaceutically acceptable salts thereof or compositions thereof can be used to reduce inflammation in a subject. In some embodiments, the inflammation can be vascular inflammation. In some embodiments, the vascular inflammation can be accompanied by atherosclerosis. In some embodiments, the vascular inflammation can be accompanied by a thromboembolic disease in the subject. In some embodiments, the vascular inflammation can be angiotensin II-induced vascular inflammation.

The compounds described herein (e.g., Compound 1) or pharmaceutically acceptable salts thereof or compositions thereof can be used in the treatment, prophylaxis, or reduction in the risk of renal disorders or dysfunctions, including end-stage renal disease, hypertension-associated renal dysfunction in a subject, kidney fibrosis, and kidney injury.

The methods of the present invention may also be used to maintain blood vessel patency, for example, in patients undergoing thrombectomy, transluminal coronary angioplasty, or in connection with vascular surgery such as bypass grafting, arterial reconstruction, atherectomy, vascular grafts, stent patency, and organ, tissue or cell implantation and transplantation. The inventive methods may be used to inhibit blood coagulation in connection with the preparation, storage, fractionation, or use of whole blood. For example, the inventive methods may be used in maintaining whole and fractionated blood in the fluid phase such as required for analytical and biological testing, e.g., for ex vivo platelet and other cell function studies, bioanalytical procedures, and quantitation of blood-containing components, or for maintaining extracorporeal blood circuits, as in a renal replacement solution (e.g., hemodialysis) or surgery (e.g., open-heart surgery, e.g., coronary artery bypass surgery). In some embodiments, the renal replacement solution can be used to treat patients with acute kidney injury. In some embodiments, the renal replacement solution can be continuous renal replacement therapy.

In addition, the methods of the present invention may be useful in treating and preventing the prothrombotic complications of cancer. The methods may be useful in treating tumor growth, as an adjunct to chemotherapy, for preventing angiogenesis, and for treating cancer, more particularly, cancer of the lung, prostate, colon, breast, ovaries, and bone.

The methods of the present invention may also include administering to a subject in need thereof an effective amount of a crystalline pharmaceutically acceptable salt of Formula (I). In some embodiments, the methods comprise dissolving the crystalline pharmaceutically acceptable salt of Formula (I) in a solvent prior to administration to the subject.

The methods of the present invention may also include administering to a subject in need thereof an effective amount of an amorphous pharmaceutically acceptable salt of Formula (I). In some embodiments, the methods comprise dissolving the amorphous pharmaceutically acceptable salt of Formula (I) in a solvent prior to administration to the subject.

### Extracorporeal Membrane Oxygenation (ECMO)

“Extracorporeal membrane oxygenation” (or “ECMO”) as used herein, refers to extracorporeal life support with a blood pump, artificial lung, and vascular access cannula, capable of providing circulatory support or generating blood flow rates adapted to support blood oxygenation, and optionally carbon dioxide removal. In venovenous ECMO, extracorporeal gas exchange is provided to blood that has been withdrawn from the venous system; the blood is then reinfused to the venous system. In venoarterial ECMO, gas exchange is provided to blood that is withdrawn from the venous system and then infused directly into the arterial system to provide partial or complete circulatory or cardiac support. Venoarterial ECMO allows for various degrees of respiratory support.

As used herein, “extracorporeal membrane oxygenation” or “ECMO” refers to extracorporeal life support that provides circulatory support or generates blood flow rates adequate to support blood oxygenation. In some embodiments, ECMO comprises removal of carbon dioxide from a subject's blood. In some embodiments, ECMO is performed using an extracorporeal apparatus selected from the group consisting of a blood pump, artificial lung, and vascular access cannula.

As used herein, “venovenous ECMO” refers to a type of ECMO in which blood is withdrawn from the venous system of a subject into an ECMO apparatus and subjected to gas exchange (including oxygenation of the blood), followed by reinfusion of the withdrawn blood into the subject's venous system. As used herein, “venoarterial ECMO” refers to a type of ECMO in which blood is withdrawn from the venous system of a subject into an ECMO apparatus and subjected to gas exchange (including oxygenation of the blood), followed by infusion of the withdrawn blood directly into the subject's arterial system. In some embodiments, venoarterial ECMO is performed to provide partial circulatory or cardiac support to a subject in need thereof. In some embodiments, venoarterial ECMO is performed to provide complete circulatory or cardiac support to a subject in need thereof.

The compounds of the present invention can be used in the treatment, prophylaxis, or reduction in the risk of a thromboembolic disorder in a subject in need thereof, wherein the subject is exposed to an artificial surface such as that of an extracorporeal membrane oxygenation (ECMO) apparatus (vide supra), which can be used as a rescue therapy in response to cardiac or pulmonary failure. The surface of an ECMO apparatus that directly contacts the subject can be a pro-thrombotic surface that can result in a thromboembolic disorder such as a venous thromboembolism, e.g., deep vein thrombosis or pulmonary embolism, leading to difficulties in treating a patient in need of ECMO. Clots in the circuit are the most common mechanical complication (19%). Major clots can cause oxygenator failure, and pulmonary or systemic emboli.

ECMO is often administered with a continuous infusion of heparin as an anticoagulant to counter clot formation. However, cannula placement can cause damage to the internal jugular vein, which causes massive internal bleeding. Bleeding occurs in 30-40% of patients receiving ECMO and can be life-threatening. This severe bleeding is due to both the necessary continuous heparin infusion and platelet dysfunction. Approximately 50% of reported deaths are due to severe bleeding complications. Aubron et al. Critical Care, 2013, 17:R73 looked at the factors associated with ECMO outcomes. The compounds of the present invention, which can be used as, for example, Factor XIa inhibitors, are thus contemplated as an alternative replacement for heparin in ECMO therapy. The compounds of the present invention are contemplated as effective agents for blocking the intrinsic pathway at plasma levels that will afford effective anti-coagulation/anti-thrombosis without marked bleeding liabilities. In some embodiments, the subject is sensitive to or has developed sensitivity to heparin. In some embodiments, the subject is resistant to or has developed resistance to heparin.

### Ischemia

“Ischemia” or an “ischemic event” is a vascular disease generally involving vascular occlusion or a restriction in blood supply to tissues. Ischemia can cause a shortage of oxygen and glucose needed for cellular metabolism. Ischemia is generally caused by problematic blood vessels that result in damage or dysfunction of tissue. Ischemia can also refer to a local loss in blood or oxygen in a given part of the body resulting from congestion (e.g., vasoconstriction, thrombosis, or embolism). Causes include embolism, thrombosis of an atherosclerosis artery, trauma, venous problems, aneurysm, heart conditions (e.g., myocardial infarction, mitral valve disease, chronic arterial fibrillation, cardiomyopathies, and prosthesis), trauma or traumatic injury (e.g., to an extremity producing partial or total vessel occlusion), thoracic outlet syndrome, atherosclerosis, hypoglycemia, tachycardia, hypotension, outside compression of a blood vessel (e.g., by a tumor), sickle cell disease, localized extreme cold (e.g., by frostbite), tourniquet application, glutamate receptor stimulation, arteriovenous malformations, rupture of significant blood vessels supplying a tissue or organ, and anemia.

A transient ischemic event generally refers to a transient (e.g., short-lived) episode of neurologic dysfunction caused by loss of blood flow (e.g., in the focal brain, spinal cord, or retinal) without acute infarction (e.g., tissue death). In some embodiments, the transient ischemic event lasts for less than 72 hours, 48 hours, 24 hours, 12 hours, 10 hours, 8 hours, 4 hours, 2 hours, 1 hour, 45 minutes, 30 minutes, 20 minutes, 15 minutes, 10 minutes, 5 minutes, 4 minutes, 3 minutes, 2 minutes, or 1 minute.

### Angioedema

Angioedema is the rapid swelling of the dermis, subcutaneous tissue, mucosa, and submucosal tissues. Angioedema is typically classified as either hereditary or acquired.

“Acquired angioedema” can be immunologic, non-immunologic, or idiopathic;

caused by e.g., allergy, as a side effect of medications, e.g., ACE inhibitor medications.

“Hereditary angioedema” or “HAE” refers to a genetic disorder that results in acute periods of edema (e.g., swelling) that may occur in nearly all parts of the body, including the face, limbs, neck, throat, larynx, extremities, gastrointestinal tract, and genitalia. Attacks of HAE can often be life-threatening, with severity depending on the area affected, e.g., abdominal attacks may result in intestinal obstruction, while swelling of the larynx and upper airway can lead to asphyxiation. Pathogenesis of hereditary angioedema may be related to unopposed activation of the contact pathway by the initial generation of kallikrein or clotting factors (e.g., Factor XII).

Signs and symptoms include swelling, e.g., of the skill of the face, mucosa of the mouth or throat, and tongue. Itchiness, pain, decreased sensation in the affected areas, urticaria (i.e., hives), or stridor of the airway may also be a sign of angioedema. However, there can be no associated itch, or urticaria, e.g., in hereditary angioedema. HAE subjects can experience abdominal pain (e.g., abdominal pain lasting one to five days, abdominal attacks increasing a subject's white blood cell count), vomiting, weakness, watery diarrhea, or rash.

Bradykinin plays an important role in angioedema, particularly hereditary angioedema. Bradykinin is released by various cell types in response to numerous different stimuli and is a pain mediator. Interfering with bradykinin production or degradation can lead to angioedema.

In hereditary angioedema, continuous production of enzyme kallikrein can facilitate bradykinin formation. Inhibition of kallikrein can interfere with bradykinin production; and treat or prevent angioedema.

The methods described herein can include those in which a subject's blood is in contact with an artificial surface. For example, in an aspect, provided herein is a method of treating a thromboembolic disorder in a subject in need thereof, the method comprising administering to the subject an effective amount of a compound represented by

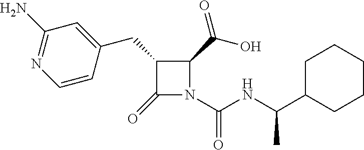 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In an aspect, provided herein is a method of reducing the risk of a thromboembolic disorder in a subject in need thereof, the method comprising administering to the subject an effective amount of a compound represented by

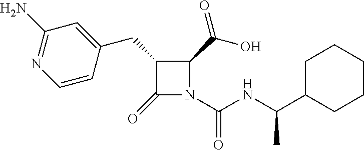 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In an aspect, provided herein is a method of prophylaxis of a thromboembolic disorder in a subject in need thereof, the method comprising administering to the subject an effective amount of a compound represented by

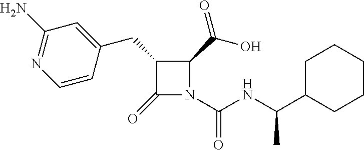 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In some embodiments of the methods provided herein, the artificial surface is in contact with blood in the subject's circulatory system. In some embodiments, the artificial surface is an implantable device, a dialysis catheter, a cardiopulmonary bypass circuit, an artificial heart valve, a ventricular assist device, a small caliber graft, a central venous catheter, or an extracorporeal membrane oxygenation (ECMO) apparatus. In some embodiments, the artificial surface causes or is associated with the thromboembolic disorder. In some embodiments, the thromboembolic disorder is a venous thromboembolism, deep vein thrombosis, or pulmonary embolism. In some embodiments, the thromboembolic disorder is a blood clot.

In some embodiments, the methods further comprise conditioning the artificial surface with a separate dose of the compound or pharmaceutically acceptable salt thereof, prior to contacting the artificial surface with blood in the circulatory system of the subject. In some embodiments, the methods further comprise conditioning the artificial surface with a separate dose of the compound or pharmaceutically acceptable salt thereof prior to or during administration of the compound or a pharmaceutically acceptable salt thereof to the subject.

In some embodiments, the methods further comprise conditioning the artificial surface with a separate dose of the compound or pharmaceutically acceptable salt thereof prior to and during administration of the compound or a pharmaceutically acceptable salt thereof to the subject.

In an aspect, provided herein is a method of treating the blood of a subject in need thereof, the method comprising administering to the subject an effective amount of a compound represented by

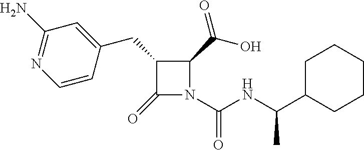 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In an aspect, provided herein is a method of maintaining the plasma level of a compound represented by

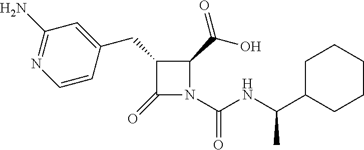 [SMILES: C[C@@H](NC(=O)N1C(=O)[C@H](Cc2ccnc(N)c2)[C@H]1C(=O)O)C1CCCCC1]

or a pharmaceutically acceptable salt thereof, in the blood of a subject in contact with an artificial surface, the method comprising:
(i) administering the compound or pharmaceutically acceptable salt thereof to the subject prior to or while contacting the artificial surface with the blood of the subject; and
(ii) conditioning an artificial surface with the compound or a pharmaceutically acceptable salt thereof prior to or while contacting the artificial surface with the blood of the subject;
   thereby maintaining the plasma level of the compound or a pharmaceutically acceptable salt thereof in the blood of the subject.

In some embodiments of the methods described herein, the compound, or a pharmaceutically acceptable salt thereof, maintains a constant activated partial thromboplastin time (aPTT) in the blood of the subject before and after contact with the artificial surface. In some embodiments, the compound or a pharmaceutically acceptable salt thereof is administered to the subject prior to and while contacting the artificial surface with the blood of the subject.

In some embodiments, the artificial surface is conditioned with the compound or a pharmaceutically acceptable salt thereof prior to and while contacting the artificial surface with the blood of the subject. In some embodiments, the method further prevents or reduces risk of a blood clot formation in the blood of the subject in contact with the artificial surface.

In some embodiments, the artificial surface is a cardiopulmonary bypass circuit. In some embodiments, the artificial surface is an extracorporeal membrane oxygenation (ECMO) apparatus. In some embodiments, the ECMO apparatus is venovenous ECMO apparatus or venoarterial ECMO apparatus.

In an aspect, provided herein is a method of preventing or reducing a risk of a thromboembolic disorder in a subject during or after a medical procedure, comprising:

(i) administering to the subject an effective amount of a compound represented by:

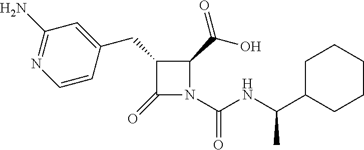 [SMILES: C[C@@H](NC(=O)N1C(=O)[C@H](Cc2ccnc(N)c2)[C@H]1C(=O)O)C1CCCCC1]

or pharmaceutically acceptable salt thereof, before, during, or after the medical procedure; and

(ii) contacting blood of the subject with an artificial surface;

thereby preventing or reducing the risk of the thromboembolic disorder during or after the medical procedure.

In some embodiments, the artificial surface is conditioned with the compound or pharmaceutically acceptable salt thereof prior to administration of the compound to the subject prior to, during, or after the medical procedure.

In some embodiments, the artificial surface is conditioned with a solution comprising the compound or a pharmaceutically acceptable salt thereof prior to administration of the compound or a pharmaceutically acceptable salt thereof to the subject prior to, during, or after the medical procedure. In some embodiments, the solution is a saline solution, Ringer's solution, or blood. In some embodiments, the solution further comprises blood. In some embodiments, the blood is acquired from the subject or a donor.

In some embodiments, the thromboembolic disorder is a blood clot.

In some embodiments, the medical procedure comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

In some embodiments, the medical procedure comprises a cardiopulmonary bypass. In some embodiments, the medical procedure comprises an oxygenation and pumping of blood via extracorporeal membrane oxygenation (ECMO). In some embodiments, the

ECMO is venovenous ECMO or venoarterial ECMO.

In some embodiments of the methods described herein, the pharmaceutically acceptable salt of the compound is a hydrochloride salt. In some embodiments, the subject is a human. In some embodiments, the subject has an elevated risk of a thromboembolic disorder. In some embodiments, the thromboembolic disorder is a result of a complication in surgery.

In some embodiments, the subject is sensitive to or has developed sensitivity to heparin. In some embodiments, the subject is resistant to or has developed resistance to heparin.

In some embodiments, the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

### Pharmaceutical Compositions

The compositions described herein include the compound described herein (e.g., Compound 1 as well as additional therapeutic agents, if present, in amounts effective for achieving the treatment of a disease or disease symptoms (e.g., such as a disease associated with Factor XIa or kallikrein). Thus in an aspect, described herein is a pharmaceutical composition comprising a crystalline pharmaceutically acceptable salt of Formula (I) and a pharmaceutically acceptable excipient. In another aspect, described herein is a pharmaceutical composition comprising an amorphous pharmaceutically acceptable salt of Formula (I) and a pharmaceutically acceptable excipient.

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions may be in the form of a solid lyophilized composition that can be reconstituted by addition of a compatible reconstitution diluent prior to parenteral administration or in the form of a frozen composition adapted to be thaws and, if desired, diluted with a compatible diluent prior to parenteral administration. In some embodiments, the pharmaceutical composition includes a powder (e.g. lyophilized composition) dissolved in aqueous medium, e.g., a saline solution, in a unit dosage IV bag or bottle at a concentration suitable for intravenous administration to a subject. In some embodiments, ingredients of a pharmaceutical composition suitable for intravenous administration are separated from each other in a single container, e.g., a powder comprising a compound described herein or a pharmaceutically acceptable salt thereof, is separated from an aqueous medium such as a saline solution. In this latter example, the various components are separated by a seal that can be broken to contact the ingredients with each other to form the pharmaceutical composition suitable for intravenous administration.

### Routes of Administration

The pharmaceutical compositions provided herewith may be administered orally, rectally, or parenterally (e.g., intravenous infusion, intravenous bolus injection, inhalation, implantation). The term parenteral as used herein includes subcutaneous, intracutaneous, intravenous (e.g., intravenous infusion, intravenous bolus injection), intranasal, inhalation, pulmonary, transdermal, intramuscular, intraarticular, intraarterial, intrasynovial, intrasternal, intrathecal, intralesional and intracranial injection or other infusion techniques. The pharmaceutical compositions provided herewith may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The pharmaceutical compositions may be in the form of a sterile injectable preparation, for example, as a sterile injectable aqueous or oleaginous solution or suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans or other similar emulsifying agents or bioavailability enhancers which are commonly used in the manufacture of pharmaceutically acceptable solid, liquid, or other dosage forms may also be used for the purposes of formulation.

The pharmaceutical compositions provided herewith may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening or flavoring or coloring or taste masking agents may be added.

The compounds described herein can, for example, be administered by injection, intravenously (e.g., intravenous infusion, intravenous bolus injection), intraarterially, subdermally, intraperitoneally, intramuscularly, or subcutaneously; or orally, buccally, nasally, transmucosally, topically with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day (e.g., by intravenous bolus injection) or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

In some embodiments, a pharmaceutical composition formulated for oral administration, subcutaneous administration, or intravenous administration is administered to a subject from 1 time per day to 6 times per day (e.g., 2 times per day or 4 times per day). In some embodiments, a pharmaceutical composition formulated for oral administration is administered to a subject from 1 time per day to 6 times per day (e.g., 2 times per day or 4 times per day) for about 3 to 9 months. In some embodiments, a pharmaceutical composition formulated for oral administration is administered to a subject from 1 time per day to 6 times per day (e.g., 2 times per day or 4 times per day) for about 1 year. In some embodiments, a pharmaceutical composition formulated for oral administration is administered to a subject from 1 time per day to 6 times per day (e.g., 2 times per day or 4 times per day) for the rest of his or her life.

In some embodiments, the compound or pharmaceutical composition is administered to the subject intravenously. In some embodiments, the compound or pharmaceutical composition is administered to the subject subcutaneously. In some embodiments, the compound or pharmaceutical composition is administered to the subject as a continuous intravenous infusion. In some embodiments, the compound is administered to the subject as a bolus. In some embodiments, the compound or pharmaceutical composition is administered to the subject as a bolus followed by a continuous intravenous infusion.

### Combinations

In carrying out the methods of the present invention, it may be desired to administer the compounds of the invention (e.g., Factor XIa or kallikrein inhibitors) in combination with each other and one or more other agents for achieving a therapeutic benefit such as antithrombotic or anticoagulant agents, anti-hypertensive agents, anti-ischemic agents, anti-arrhythmic agents, platelet function inhibitors, and so forth. For example, the methods of the present invention may be carried out by administering the small molecule Factor XIa or kallikrein inhibitors in combination with a small molecule Factor XIa or kallikrein inhibitor. More particularly, the inventive methods may be carried out by administering the small molecule Factor XIa or kallikrein inhibitors in combination with aspirin, clopidogrel, ticlopidine or CS-747, warfarin, low molecular weight heparins (such as LOVENOX), GPIIb/GPIIIa blockers, PAI-1 inhibitors such as XR-330 and T-686, P2Y1 and P2Y12 receptor antagonists; thromboxane receptor antagonists (such as ifetroban), prostacyclin mimetics, thromboxane A synthetase inhibitors (such as picotamide), serotonin-2-receptor antagonists (such as ketanserin); compounds that inhibit other coagulation factors such as FVII, FVIII, FIX, FX, prothrombin, TAFI, and fibrinogen, or other compounds that inhibit FXI or kallikrein; fibrinolytics such as TPA, streptokinase, PAI-1 inhibitors, and inhibitors of α-2-antiplasmin such as anti-α-2-antiplasmin antibody fibrinogen receptor antagonists, inhibitors of α-1-antitrypsin, hypolipidemic agents, such as HMG-CoA reductase inhibitors (e.g., pravastatin, simvastatin, atorvastatin, fluvastatin, cerivastatin, AZ4522, and itavastatin), and microsomal triglyceride transport protein inhibitors (such as disclosed in U.S. Pat. Nos. 5,739,135, 5,712,279 and 5,760,246); antihypertensive agents such as angiotensin-converting enzyme inhibitors (e.g., captopril, lisinopril or fosinopril); angiotensin-II receptor antagonists (e.g., irbesartan, losartan or valsartan); ACE/NEP inhibitors (e.g., omapatrilat and gemopatrilat); or β-blockers (such as propranolol, nadolol and carvedilol). The inventive methods may be carried out by administering the small molecule Factor XIa or kallikrein inhibitors in combination with anti-arrhythmic agents such as for atrial fibrillation, for example, amiodarone or dofetilide. The inventive methods may also be carried out in combination continuous renal replacement therapy for treating, e.g., acute kidney injury.

In carrying out the methods of the present invention, it may be desired to administer the compounds of the invention (Factor XIa or kallikrein inhibitors) in combination with agents that increase the levels of cAMP or cGMP in cells for a therapeutic benefit. For example, the compounds of the invention may have advantageous effects when used in combination with phosphodiesterase inhibitors, including PDE1 inhibitors (such as those described in Journal of Medicinal Chemistry, Vol. 40, pp. 2196-2210 [1997]), PDE2 inhibitors, PDE3 inhibitors (such as revizinone, pimobendan, or olprinone), PDE4 inhibitors (such as rolipram, cilomilast, or piclamilast), PDE7 inhibitors, or other PDE inhibitors such as dipyridamole, cilostazol, sildenafil, denbutyline, theophylline (1,2-dimethylxanthine), ARIFLOT™ (i.e., cis-4-cyano-4-[3-(cyclopenlylox-y)-4-methoxyphenyl]cyclohexane-1-carboxylic acid), arofyline, roflumilast, C-11294A, CDC-801, BAY-19-8004, cipamfylline, SCH351591, YM-976, PD-189659, mesiopram, pumafentrine, CDC-998, IC-485, and KW-4490.

The inventive methods may be carried out by administering the compounds of the invention in combination with prothrombolytic agents, such as tissue plasminogen activator (natural or recombinant), streptokinase, reteplase, activase, lanoteplase, urokinase, prourokinase, anisolated streptokinase plasminogen activator complex (ASPAC), animal salivary gland plasminogen activators, and the like.

The inventive methods may be carried out by administering the compounds of the invention in combination with β-adrenergic agonists such as albuterol, terbutaline, formoterol, salmeterol, bitolterol, pilbuterol, or fenoterol; anticholinergics such as ipratropium bromide; anti-inflammatory cortiocosteroids such as beclomethasone, triamcinolone, budesonide, fluticasone, flunisolide or dexamethasone; and anti-inflammatory agents such as cromolyn, nedocromil, theophylline, zileuton, zafirlukast, monteleukast and pranleukast.

Small molecule Factor XIa or kallikrein inhibitors may act synergistically with one or more of the above agents. Thus, reduced doses of thrombolytic agent(s) may be used, therefore obtaining the benefits of administering these compounds while minimizing potential hemorrhagic and other side effects.

### Course of Treatment

The compositions described herein include an effective amount of a compound of the invention (e.g., a Factor XIa or kallikrein inhibitor) in combination and one or more other agents (e.g., an additional therapeutic agent) such as antithrombotic or anticoagulant agents, anti-hypertensive agents, anti-ischemic agents, anti-arrhythmic agents, platelet function inhibitors, and so forth for achieving a therapeutic benefit.

In some embodiments, the additional therapeutic agent is administered following administration of the compound of the invention (e.g., a Factor XIa or kallikrein inhibitor). In some embodiments, the additional therapeutic agent is administered 15 minutes, 30 minutes, 1 hour, 2 hours, 4 hours, 6 hours, 8 hours, 10 hours, 12 hours, 14 hours, 18 hours, 24 hours, 48 hours, 72 hours or longer after administration of the compound of the invention (e.g., a Factor XIa or kallikrein inhibitor). In some embodiments, the additional therapeutic agent is administered (e.g., orally) after discharge from a medical facility (e.g., a hospital).

In some embodiments, the compound of the invention (e.g., a Factor XIa or kallikrein inhibitor) and the additional therapeutic agent are co-formulated into a single composition or dosage. In some embodiments, the compound of the invention (e.g., a Factor XIa or kallikrein inhibitor) and the additional therapeutic agent are administered separately. In some embodiments, the compound of the invention (e.g., a Factor XIa or kallikrein inhibitor) and the additional therapeutic agent are administered sequentially. In some embodiments, the compound of the invention (e.g., a Factor XIa or kallikrein inhibitor) and the additional therapeutic agent are administered separately and sequentially. In general, at least one of the compound of the invention (e.g., a Factor XIa or kallikrein inhibitor) and the additional therapeutic agent is administered parenterally (e.g., intranasally, intramuscularly buccally, inhalation, implantation, transdermal, intravenously (e.g., intravenous infusion, intravenous bolus injection), subcutaneous, intracutaneous, intranasal, pulmonary, transdermal, intraarticular, intraarterial, intrasynovial, intrasternal, intrathecal, intralesional and intracranial injection or other infusion techniques); orally; or rectally, for example, intramuscular injection or intravenously (e.g., intravenous infusion, intravenous bolus injection)). In some embodiments, compound of the invention is administered parenterally (e.g., intranasally, buccally, intravenously (e.g., intravenous infusion, intravenous bolus injection) or intramuscularly). In some embodiments, the additional therapeutic agent is administered orally. In some embodiments, the compound of the invention (e.g., a Factor XIa or kallikrein inhibitor) is administered parenterally (e.g., intranasally, buccally, intravenously (e.g., intravenous infusion, intravenous bolus injection) or intramuscularly) and the additional therapeutic agent is administered orally.

In some embodiments, the compound of the invention (e.g., a Factor XIa or kallikrein inhibitor) may be administered once or several times a day. A duration of treatment may follow, for example, once per day for a period of about 1, 2, 3, 4, 5, 6, 7 days or more. In some embodiments, the treatment is chronic (e.g., for a lifetime). In some embodiments, either a single dose in the form of an individual dosage unit or several smaller dosage units or by multiple administrations of subdivided dosages at certain intervals is administered. For instance, a dosage unit can be administered from about 0 hours to about 1 hr, about 1 hr to about 24 hr, about 1 to about 72 hours, about 1 to about 120 hours, or about 24 hours to at least about 120 hours post injury. Alternatively, the dosage unit can be administered from about 0.5, 1, 1.5, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours or longer post injury. Subsequent dosage units can be administered any time following the initial administration such that a therapeutic effect is achieved. In some embodiments, the initial dose is administered orally. In some embodiments, doses subsequent to the initial dose are administered parenterally (e.g., intranasally, intramuscularly buccally, inhalation, implantation, transdermal, intravenously (e.g., intravenous infusion, intravenous bolus injection), subcutaneous, intracutaneous, intranasal, pulmonary, transdermal, intraarticular, intraarterial, intrasynovial, intrasternal, intrathecal, intralesional and intracranial injection or other infusion techniques); orally; or rectally.

In some embodiments, compounds of the invention (e.g., a Factor XIa or kallikrein inhibitor) is administered orally, e.g., as an liquid or solid dosage form for ingestion, for about 5 minutes to about 1 week; about 30 minutes to about 24 hours, about 1 hour to about 12 hours, about 2 hours to about 12 hours, about 4 hours to about 12 hours, about 6 hours to about 12 hours, about 6 hours to about 10 hours; about 5 minutes to about 1 hour, about 5 minutes to about 30 minutes; about 12 hours to about 1 week, about 24 hours to about 1 week, about 2 days to about 5 days, or about 3 days to about 5 days. In one embodiment, the compound of the invention (e.g., a Factor XIa or kallikrein inhibitor) is administered orally as a liquid dosage form. In another embodiment, the compound of the invention (e.g., a Factor XIa or kallikrein inhibitor) is administered orally as a solid dosage form.

Where a subject undergoing therapy exhibits a partial response, or a relapse following completion of the first cycle of the therapy, subsequent courses of therapy may be needed to achieve a partial or complete therapeutic response (e.g., chronic treatment, e.g., for a lifetime).

In some embodiments, the compound of the invention (e.g., a Factor XIa or kallikrein inhibitor) is administered intravenously, e.g., as an intravenous infusion or intravenous bolus injection, for about 5 minutes to about 1 week; about 30 minutes to about 24 hours, about 1 hour to about 12 hours, about 2 hours to about 12 hours, about 4 hours to about 12 hours, about 6 hours to about 12 hours, about 6 hours to about 10 hours; about 5 minutes to about 1 hour, about 5 minutes to about 30 minutes; about 12 hours to about 1 week, about 24 hours to about 1 week, about 2 days to about 5 days, or about 3 days to about 5 days. In one embodiment, the compound of the invention (e.g., a Factor XIa or kallikrein inhibitor) is administered as an intravenous infusion for about 5, 10, 15, 30, 45, or 60 minutes or longer; about 1, 2, 4, 6, 8, 10, 12, 16, or 24 hours or longer; about 1, 2, 3, 4, 5, 6, 7, 8, 9, or 10 days or longer.

### Dosages and Dosing Regimens

The effective amount of a small molecule Factor XIa or kallikrein inhibitor administered according to the present invention may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### EXAMPLES

In order that the invention described herein may be more fully understood, the following examples are set forth. Starting materials and various intermediates described in the following examples may be obtained from commercial sources, prepared from commercially available organic compounds, or prepared using known synthetic methods. The examples described in this application are offered to illustrate the compounds provided herein and are not to be construed in any way as limiting their scope.

### General Procedures

All non-aqueous reactions were run under an atmosphere of nitrogen to maintain an anhydrous atmosphere and to maximize yields. All reactions were stirred using an overhead stirring assembly or magnetically, with the aid of a Teflon-coated stir bar. The description ‘drying over’ refers to drying of a reaction product solution over a specified drying agent and then filtration of the solution though a suitable filter paper or through a sintered glass funnel.

The descriptions ‘was concentrated’, ‘was concentrated at reduced pressure’, or ‘evaporated’ refers to removal of solvents under reduced pressure using a rotary evaporator. Unless otherwise specified, proton NMR spectra (1H) are measured at 400 MHz in the specified solvent.

Abbreviations used in the experimental examples are listed in the Abbreviations Table below.

Abbreviation Table

| | |
|---|---|
| MeCN | Acetonitrile |
| DCM | Dichloromethane |
| Ether | Diethyl ether |
| hr | Hours |
| HPLC | High-performance liquid chromatography |
| IPA | Isopropyl alcohol |
| min | Minutes |
| TBME | Methyl tert-butyl ether |
| NMR | Nuclear magnetic resonance instrument |
| RT | Room temperature |
| TFA | Trifluoroacetic acid |
| THF | Tetrahydrofuran |
| Concentrated or | Concentration of organic solutions under |
| concentrated | reduced pressure with the use of a |
| in vacuo | rotary evaporator |

### Example 1: Exemplary Synthesis of Compound 1.HCl and Exemplary Preparation of Single Crystals of Compound 1.HCl

### A) Exemplary Synthesis of Compound 1.HCl:

A non-limiting example of the synthesis of (2S,3R)-3-[(2-aminopyridin-4-yl)methyl]-1-{[(1R)-1-cyclohexylethyl]carbamoyl}-4-oxoazetidine-2-carboxylic acid trifluoroacetate (structure 2 below) can be found in U.S. Pat. No. 9,499,532, which is incorporated herein by reference.

Synthesis of Compound 1.HCl from 2

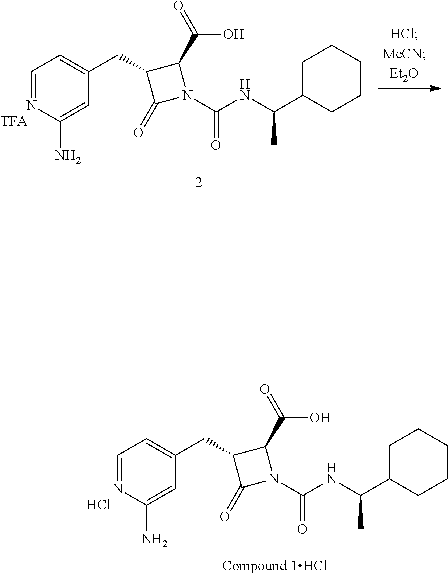 [SMILES: C[C@@H](NC(=O)N1C(=O)[C@H](Cc2ccnc(N)c2)[C@H]1C(=O)O)C1CCCCC1 ; Cl ; C[C@@H](NC(=O)N1C(=O)[C@H](Cc2ccnc(N)c2)[C@H]1C(=O)O)C1CCCCC1]

Acetonitrile (12 mL) was added to (2S,3R)-3-[(2-aminopyridin-4-yl)methyl]-1-{[(1R)-1-cyclohexylethyl]carbamoyl}-4-oxoazetidine-2-carboxylic acid trifluoroacetate (1.23 g, 2.52 mmol, structure 2) giving a hazy solution. The MeCN layer was extracted twice with hexanes (2×12 mL) The MeCN solution was clarified through a syringe filter, and the solution was concentrated to 6 mL giving a suspension. Concentrated HCl (0.42 mL, 2 equiv) was added. Ether (12 mL) was added with additional precipitation occurring, so then a total of 24 mL of ether were added. The suspension was cooled to 1° C. for 15 min. The solid was isolated by filtration through a medium fritted funnel with a cold ether rinse. The solid was air-dried to give Compound 1.HCl (0.82 g, 79% yield).

Multiple batches of Compound 1.HCl were combined and treated with ether (30 mL) giving a partially stirrable slurry. A total of 45 mL of Et₂O were needed to yield a stirrable slurry. The mixture was filtered on a medium fritted funnel after 10 min with two Et₂O (5 mL) washes. The white solid was dried at 50° C. in a vacuum oven overnight to give pure Compound 1.HCl.

¹H NMR (400 MHz, CD₃OD) ppm δ 7.79 (1 H, d, J=6.8 Hz), 6.99 (1 H, s), 6.90 (1 H, dd, J=1.5, 6.8 Hz), 6.61 (1H, d J=8.8), 4.28 (1H, d, J=2.8) 3.70 (2H, m), 3.23 (2H, m) 1.75 (5H, m) 1.40 (1H, m) 1.25 (3H, m) 1.15 (3H, d, J=6.8 Hz) 1.00 (2H, m). HPLC: Zorbax 50 mm; flow=1.5 mL/min; 240 nm; temp=at 30° C.; A=1 mL TFA/1L water; B=2.8 mL TFA/4 L MeCN; A time=0, A:B=95:5; Go to 2:98 A:B over 6 min; go to A:B=95:5 from 6 to 7 min. Compound 1.HCl Retention Time: 3.21.

### B) Exemplary Preparation of Compound 1.HCl Single Crystals:

The single crystals of Compound 1.HCl were isolated by adding 50 mg of Compound 1.HCl in 2 mL of MeCN and 0.2 mL of water and dissolving at 40° C. After a clear solution was left for evaporation at room temperature in the hood. 5 mg of seed crystals (Compound 1.HCl) were added to the standing solution and left undisturbed until crystals were observed the following day.

### Example 2: Details of the Analytical Techniques

Differential Scanning calorimetry (DSC)

DSC data were collected using a TA Instruments Q10 DSC. Approximately, samples (2-8 mg) were placed in unsealed but covered hermetic alodined aluminum sample pans and scanned from 30 to 300° C. at a rate of 10° C./min under a nitrogen purge of 50 mL/min.

### Thermal Gravimetric Analysis (TGA)

TGA data were collected using a TA Instruments TGA Q500. Approximately, 5-10 mg samples were placed in an open, pre-tared aluminum sample pan and scanned from 25 to 300° C. at a rate of 10° C./min using a nitrogen purge at 60 mL/min.

### X-Ray Powder Diffractometer (XRPD)

X-ray powder diffraction patterns were obtained using a Bruker D8 Advance equipped with a Cu Kα radiation source (λ=1.54° A), a 9-position sample holder and a LYNXEYE super speed detector. Samples were placed on zero-background, silicon plate holders for analysis.

### Dynamic Vapor Sorption (DVS)

Samples were analyzed using an Aquadyne DVS-2 gravimetric water sorption analyzer. The relative humidity was adjusted between 2-95% and the weight of the sample was continuously monitored and recorded with respect to the relative humidity and time.

### Proton Nuclear Magnetic Resonance (¹H-NMR)

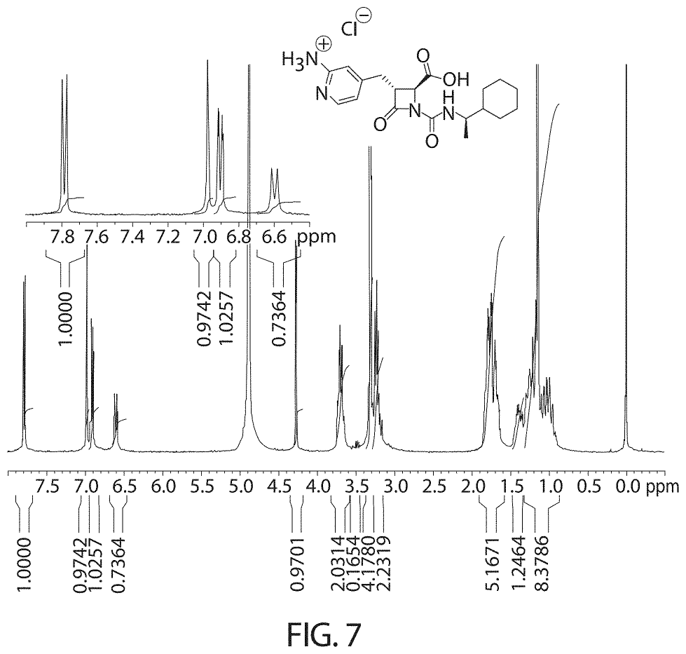

Samples were prepared by dissolving the compound in deuterated dimethylsulfoxide with 0.05% (v/v) tetramethylsilane (TMS). Spectra as shown in FIGS. 7, 13, and 16 were collected at ambient temperature on a Bruker Avance 300 MHz NMR equipped with TopSpin software, and the number of scans was 16.

### Karl Fischer (KF)

The apparent water content in samples was determined by Karl Fischer titration using a Mettler Toledo DL39 Coulometric KF Titrator. HYDRANAL-Coulomat AD was used as the titrant. About 20 mg of the solid was used for titration. The analytical parameters are presented in the following.

| KF Parameter | Value |
|---|---|
| Speed [%] | 40 |
| Mix time [sec] | 10 |
| Auto start | No |
| Blank [μg] | 0 |
| Drift [μg/min] | 5 |
| Calculation | Ug |
| Standby | Yes |
| Initial drift [μg/min] | <10 |
| Initial Potential [mV] | 100 |

### Optical Microscopy

Samples were analyzed using an Olympus BX53 polarized light microscope equipped with a PAXcam 3 digital microscope camera.

### Example 3: Baseline Characterization of Compound 1.HCl

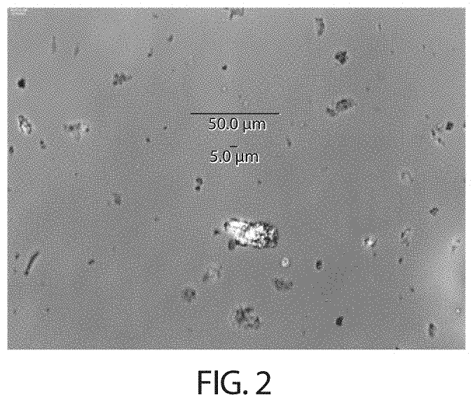

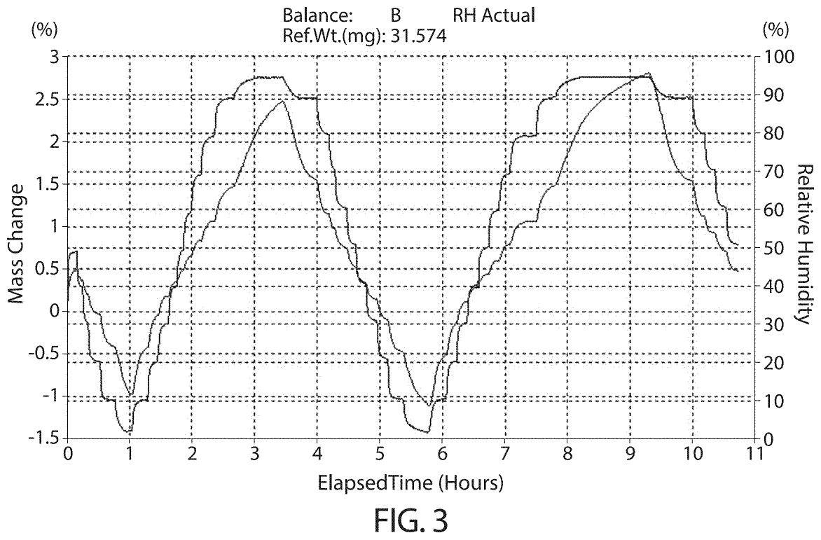

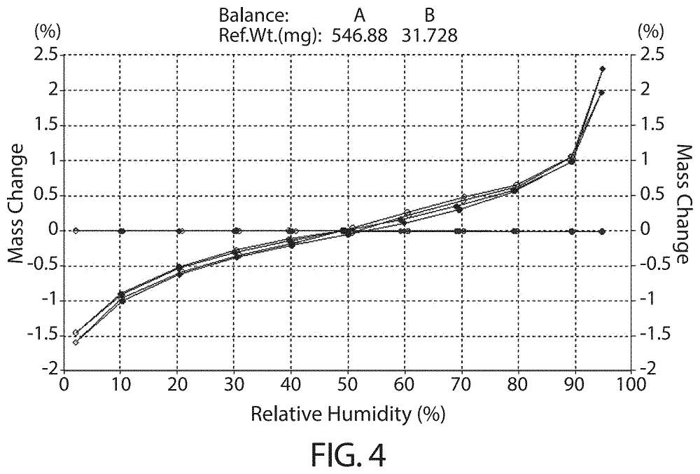

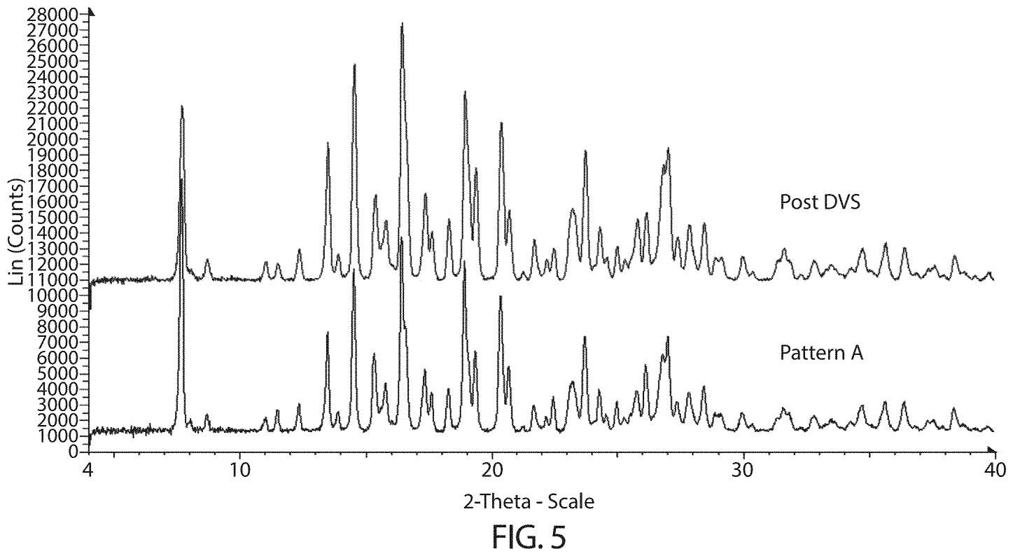

Compound 1.HCl was characterized using X-ray powder diffraction (XRPD) and optical microscopy as illustrated in FIGS. 1 and 2, respectively. The same material was subjected under the Dynamic Vapor Sorption (DVS, FIGS. 3 and 4). It suggests about 4% moisture uptake of the material when exposed to relative humidity levels between 0-95%. After DVS the XRPD indicated the crystalline pattern did not change (FIG. 5, still Pattern A).

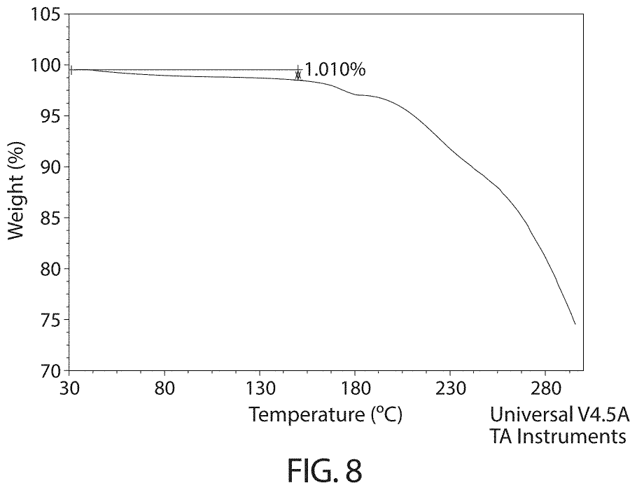

DSC analysis indicated potential water loss at 84° C. and an endothermic thermal event at 192° C. (FIG. 6), ¹H-NMR was examined (FIG. 7) in d₄-MeOD confirming the structure, the TGA showed approximately 1.0% weight loss at 150° C. (FIG. 8) and KF showed 1.5 weight % of water in the received material.

### Example 4: Solubility Assessment of Compound 1.HCl

Solubility of Compound 1.HCl was measured gravimetrically in 12 different solvents and solvent mixtures at 15 and 45° C. About 90 mg of the compound was dispensed in 1 mL of the solvent/solvent mixture and slurried for 48 h.

The vials were centrifuged. The supernatant was collected and left for slow evaporation under vacuum at 45° C. The solids obtained after evaporation were used to determine the solubility of Compound 1.HCl and analyzed by XRPD for any new form. Table 1 represents the solubility of Compound 1.HCl in different solvents.

TABLE 1
Solubility of Compound 1•HCl in different solvent/solvent
mixtures at 15° C. and 45° C.

| | | Temperature | Solubility |
|---|---|---|---|
| Sample ID | Solvent | (° C.) | (mg/mL)* |
| 1 | Water | 15 | >100 |
| 2 | | 45 | >100 |
| 3 | TBME | 15 | 0.4 |
| 4 | | 45 | 4.1 |
| 5 | Heptane | 15 | <1 |
| 6 | | 45 | 2.1 |
| 7 | IPA | 15 | 17.0 |
| 8 | | 45 | 43.0 |
| 9 | EtOAc | 15 | 0.2 |
| 10 | | 45 | 2.1 |
| 11 | Acetone | 15 | 2.2 |
| 12 | | 45 | 4.8 |
| 13 | EtOH:H₂O (95:5) | 15 | >100 |
| 14 | | 45 | >100 |
| 15 | MeOH:H₂O (1:1) | 15 | >100 |
| 16 | | 45 | >100 |
| 17 | MeOH | 15 | >100 |
| 18 | | 45 | >100 |
| 19 | IPA:H₂O (9:1) | 15 | >100 |
| 20 | | 45 | >100 |
| 21 | THF | 15 | 1.2 |
| 22 | | 45 | 6.7 |
| 23 | MeCN | 15 | 0.7 |
| 24 | | 45 | 0.7 |

*Determined gravimetrically

The precipitates from all the slurry experiments and the solids obtained after slow evaporation were analyzed by XRPD and the results are enlisted in Table 2.

TABLE 2
Summary of XRPD analysis of slurry and slow evaporation experiments.

| | | | | XRPD - Slow |
|---|---|---|---|---|
| Sample | Temperature | | XRPD - | evaporation |
| ID | (° C.) | Solvent | Slurry | (45° C.) |
| 1 | 15 | Water | N/A* | Pattern A |
| 2 | 45 | | N/A* | Pattern A |
| 3 | 15 | TBME | Pattern A | Not enough solid |
| 4 | 45 | | Pattern A | Not enough solid |
| 5 | 15 | Heptane | Pattern A | Not enough solid |
| 6 | 45 | | Pattern A | Not enough solid |
| 7 | 15 | IPA | Pattern A | Pattern A |
| 8 | 45 | | Pattern A | Pattern A |
| 9 | 15 | EtOAc | Pattern A | Not enough solid |
| 10 | 45 | | Pattern A | Not enough solid |
| 11 | 15 | Acetone | Pattern A | Not enough solid |
| 12 | 45 | | Pattern A | Not enough solid |
| 13 | 15 | EtOH:H₂O | N/A* | Pattern A |
| 14 | 45 | (95:5) | N/A* | Pattern A |
| 15 | 15 | MeOH:H₂O | N/A* | Pattern A |
| 16 | 45 | (1:1) | N/A* | Amorphous |
| 17 | 15 | MeOH | N/A* | Pattern A |
| 18 | 45 | | N/A* | Semi-crystalline |
| 19 | 15 | IPA:H₂O | N/A* | Pattern A |
| 20 | 45 | (9:1) | N/A* | Pattern A |
| 21 | 15 | THF | Pattern A | Not enough solid |
| 22 | 45 | | Pattern A | Not enough solid |
| 23 | 15 | MeCN | Pattern A | Not enough solid |
| 24 | 45 | | Pattern A | Not enough solid |

*Clear solution was obtained.

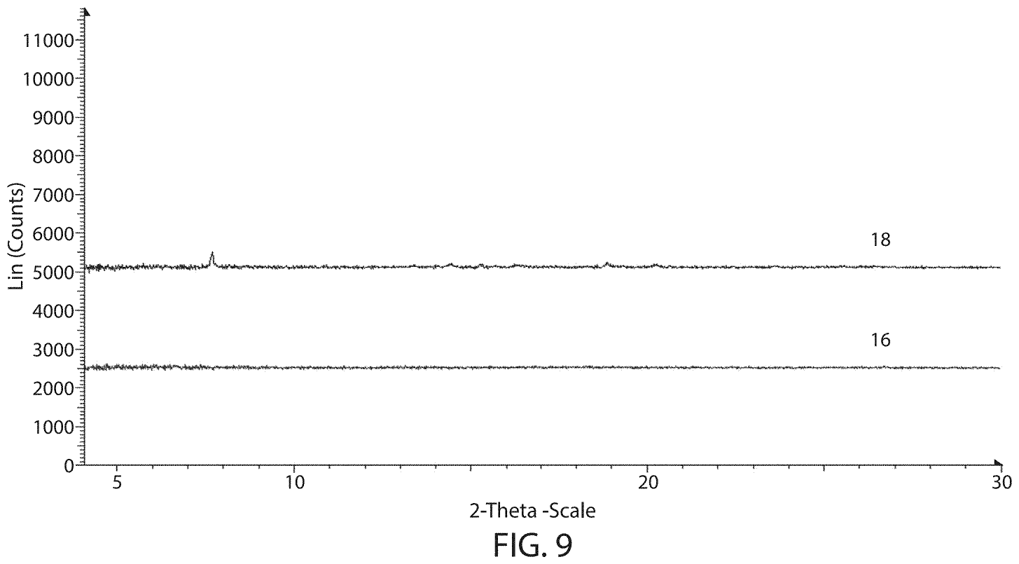

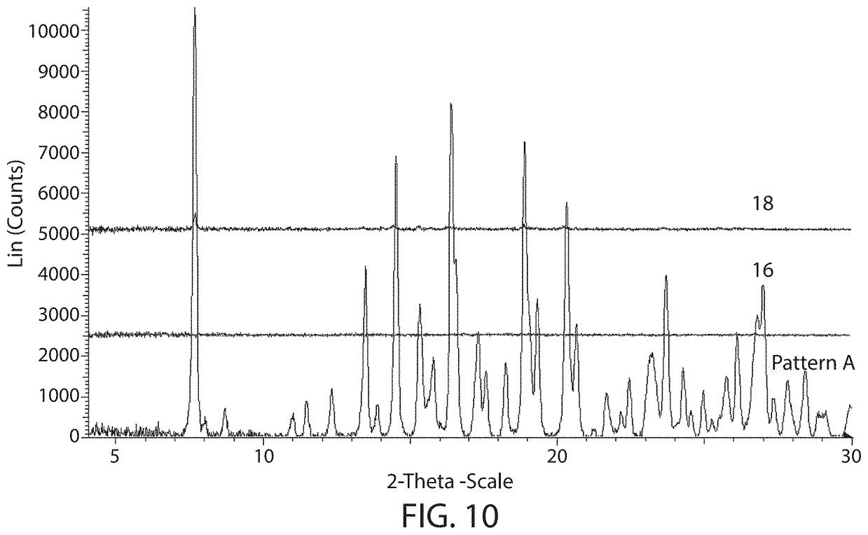

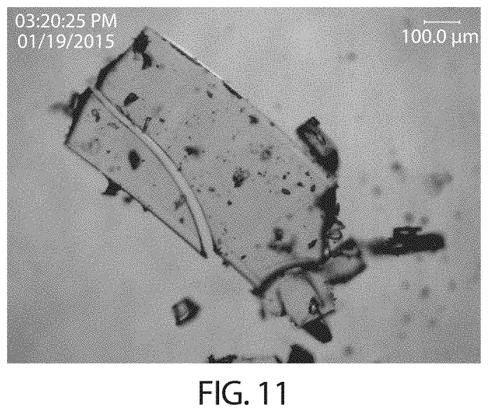

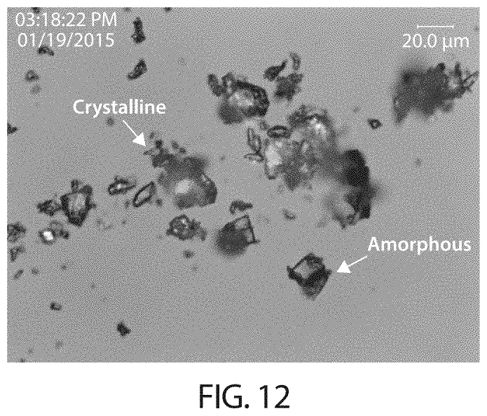

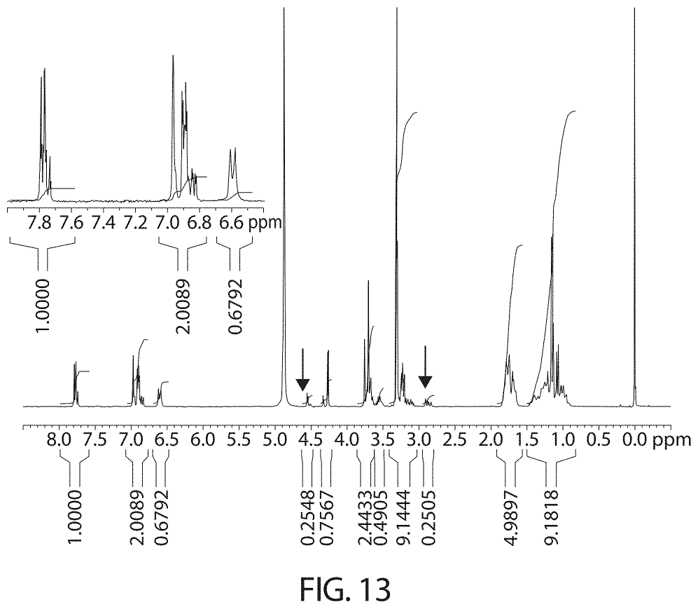

The samples 16 and 18 were analyzed by XRPD and optical microscope. FIGS. 9 and 10 illustrate the XRPD patterns of the samples. FIGS. 11 and 12 illustrate the microscopic images of the semi-crystalline material obtained after evaporation. The amorphous form (sample ID 16) was further characterized by ¹H-NMR and DSC. The ¹H-NMR spectrum of the amorphous form was consistent with Compound 1.HCl. However, two additional peaks at 4.5 ppm and 3.85 ppm were observed that could be possible impurities (indicated in FIG. 13). The DSC (FIG. 14) of the amorphous revealed a broad endotherm at around 105° C. followed by another endotherm at around 187° C. (MP of Compound 1.HCl).

### Example 5: Cooling Crystallization of Compound 1.HCl

### Room Temperature Cooling Crystallization Experiments

Cooling crystallization experiments of Compound 1.HCl in six different solvents were performed to screen for new polymorphs. A known amount of Compound 1.HCl (see Table 3) was dissolved in the given volume of the solvent at 55° C. If the solids did not dissolve, the solution filtered and the supernatant was evaporated at room temperature over the weekend. Table 3 summarizes the experimental details and the results of XRPD analysis on the material obtained after evaporation.

TABLE 3
Experimental details of room temperature cooling crystallization.

| Sample ID | Solvent, volume (mL) | Amount of API, mg | Result |
|---|---|---|---|
| A3 | TBME, 3 | 20 | No solid were |
| | | | obtained |
| A4 | Acetone, 3 | 20 | Pattern A |
| A5 | THF, 3 | 20 | Pattern A |
| A6 | IPA, 2 | 50 | Pattern A |
| A7 | Water, 1 | 100 | No crystals |
| A8 | MeOH, 1 | 100 | Pattern A |

### Low Temperature Cooling Crystallization Experiments

Similarly, low temperature (5° C.) cooling crystallization experiments of Compound 1.HCl in six different solvents were performed. A known amount of Compound 1.HCl (see Table 4) was dissolved in the given volume of the solvent at 55° C. If the solids did not, the solution filtered and the supernatant was evaporated at room temperature over the weekend. Table 4 summarizes the experimental details and the results of XRPD analysis on the material obtained after evaporation.

TABLE 4
Experimental details of low temperature cooling crystallization.

| Sample ID | Solvent, volume (mL) | Amount of API, mg | Result |
|---|---|---|---|
| B1 | TBME:MeOH (8:2), 2.4 | 20 | Pattern A |
| B2 | Acetone:H₂O (8:2), 1 | 20 | No crystals |
| B3 | THF:H₂O (8:2), 1 | 20 | No crystals |
| B4 | IPA, 2 | 50 | No crystals |
| B5 | Water, 1 | 100 | No crystals |
| B6 | MeOH, 1 | 100 | Pattern A |

### Example 6: Anti-Solvent Addition Experiments of Compound 1.HCl

Anti-solvent addition experiments for Compound 1.HCl were performed by using several anti-solvents. Of the 12 experiments, 8 of the samples resulted in the precipitation of solids while four of the experiments did not yield any solids. Table 5 summarizes the experimental details and the results.

TABLE 5
Experimental details of anti-solvent addition experiments.

| Sample | Amount of | | | |
|---|---|---|---|---|
| ID | API | Solvent | Anti-solvent | Result |
| B7 | 50 mg | IPA, 1 mL | THF, 2 mL | Clear solution |
| B8 | 50 mg | IPA, 1 mL | MeCN, 2 mL | Clear solution |
| B9 | 50 mg | Water, 0.5 mL | THF, 2 mL | Clear solution |
| B10 | 50 mg | Water, 0.5 mL | MeCN, 2 mL | Clear solution |
| B11 | 50 mg | MeOH, 0.5 mL | EtOAc, 2 mL | Pattern A |
| B12 | 50 mg | MeOH, 0.5 mL | TBME, 2 mL | Pattern A |
| B13 | 50 mg | IPA, 0.5 mL | EtOAc, 2 mL | Pattern A |
| B14 | 50 mg | IPA, 0.5 mL | TBME, 2 mL | Pattern A |
| B15 | 50 mg | MeOH, 0.5 mL | Heptane, 3 mL | Pattern A |
| B16 | 50 mg | IPA, 1 mL | Heptane, 3 mL | Pattern A |
| B17 | 50 mg | Water, 0.5 mL | Acetone, 3 mL | Pattern A |
| B18 | 50 mg | IPA, 1 mL, | Acetone, 3 mL | Pattern A |

### Example 7: Characterization of Amorphous Compound 1.HCl

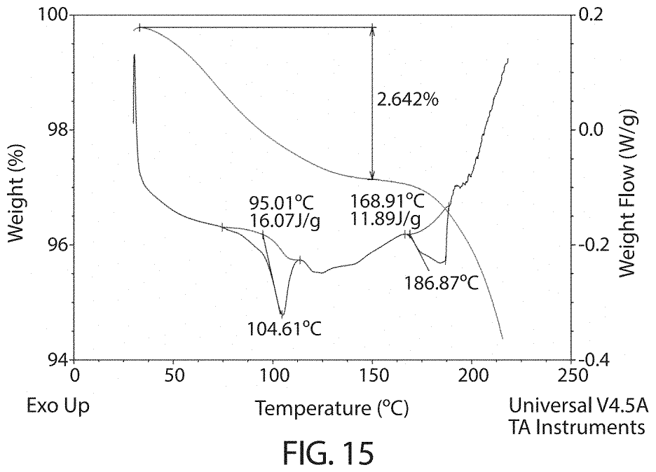

The amorphous Compound 1.HCl was scaled-up by dissolving 500 mg of the material in 6 mL of MeOH:H₂O (1:1) and drying at 45° C. under vacuum (Sample ID: C1). The amorphous form was further characterized DSC, TGA, optical microscope, Karl Fisher, ¹H-NMR and DVS. FIG. 15 illustrates the DSC thermogram and the TGA overlay of amorphous Compound 1.HCl. From the thermal analysis it was observed that the amorphous salt undergoes a weight loss from 30 to 105° C. The first endotherm in the DSC thermogram represents the possible water loss followed by possible form transformation (105-150° C.) and the second endotherm corresponds to the melting point of Pattern A.

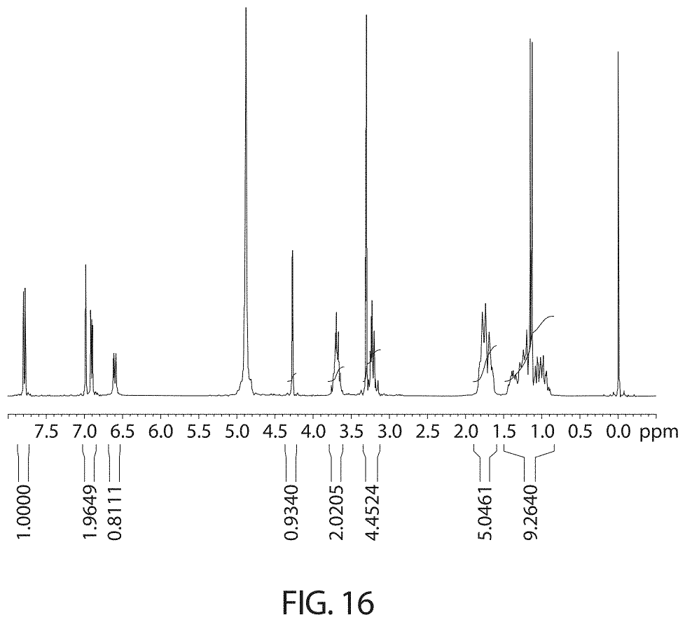

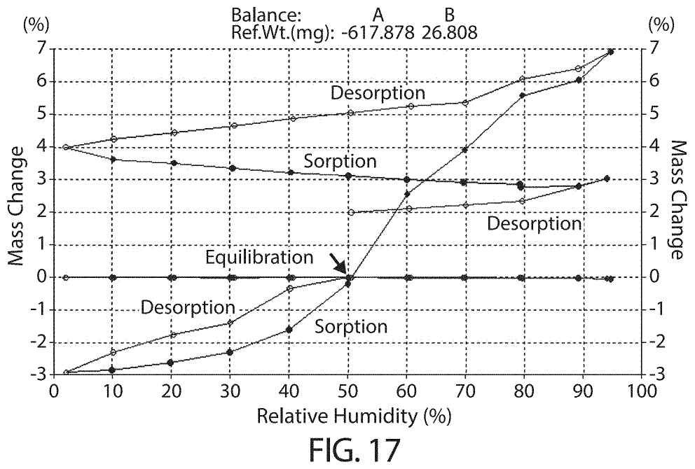

The water content by Karl Fischer was found to be around 2.58% in the amorphous sample. The purity of the sample was also verified by ¹H-NMR. The impurities which were observed in the first experiment were not observed in the scale-up experiment (FIG. 16). The amorphous salt was also studied by DVS. During Desorption 1, (from 50 to 0% RH), a weight loss of around 3% was observed whilst, during Sorption 1 (from 0 to 95% RH), a weight gain of 10%. Desorption 2 indicates a weight loss of 3% was observed and during Sorption 2 (from 0 to 95% RH) the weight loss was continued to additional 1%. In the final stage, Desorption 3 (from 95 to 50% RH), around 1% weight loss was observed (see FIG. 17). The amorphous Compound 1.HCl was analyzed by XRPD and optical microscopy post-DVS experiment. The XRPD analysis revealed that the amorphous form reverts back to crystalline salt (Pattern A).

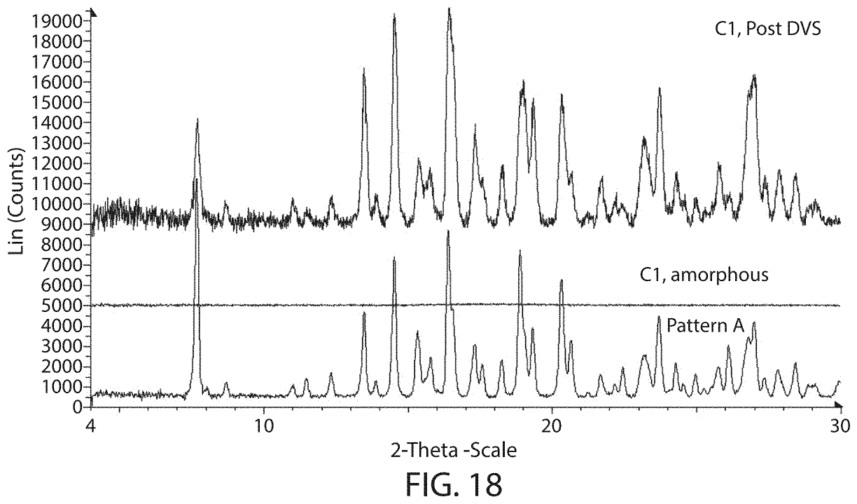

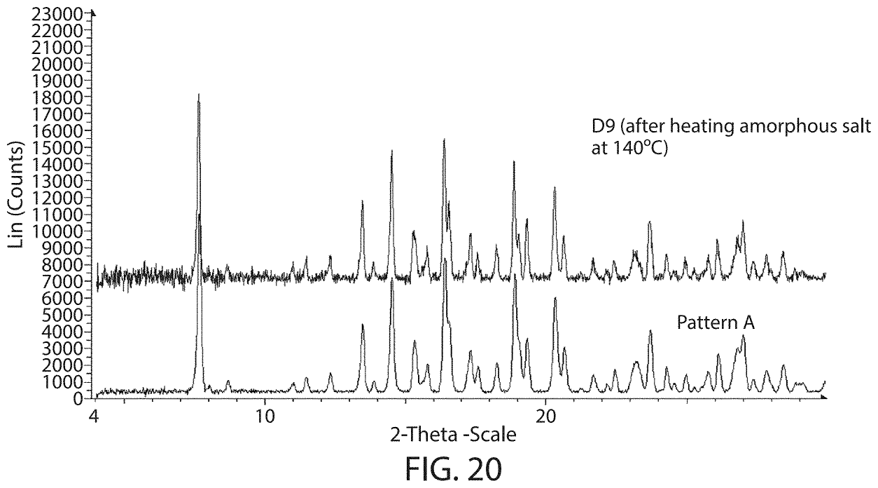

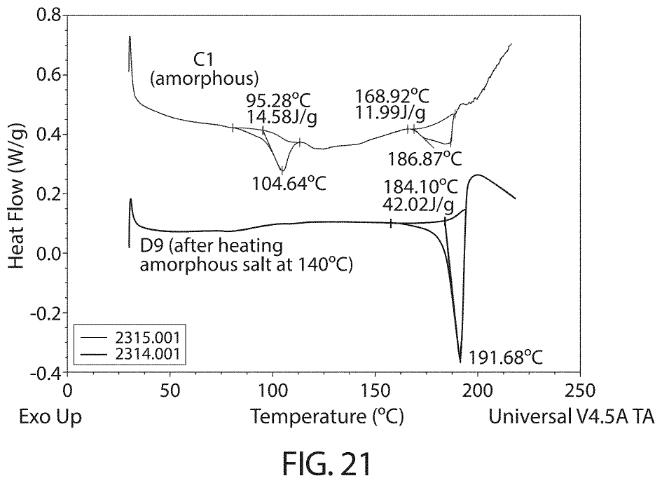

FIG. 18 illustrates the post-DVS XRPD comparison with the amorphous and Pattern A (original salt). The amorphous salt was heated to 140° C. for 30 min and characterized by XRPD and DSC (Sample ID: D9). The XRPD analysis of the heated sample revealed the transformation to Pattern A (crystalline salt) as illustrated in FIG. 20. The DSC thermogram in FIG. 21 also confirms the conversion of amorphous to crystalline (Pattern A) after heating the amorphous sample at 140° C.

### Example 8: Neat and Solvent Drop Grinding of Compound 1.HCl

Neat grinding experiments of Compound 1.HCl and solvent drop grinding (40 μL) experiments were carried out by grinding 20-25 mg of the salt in a mortar and pestle for 5 minutes and were analyzed by XRPD (Table 6).

TABLE 6
Summary of neat and solvent drop grinding.

| Sample ID | Solvent used for grinding | Result (XRPD analysis) |
|---|---|---|
| D1 | MeOH:H₂O (1:1) | Semi crystalline |
| D2 | MeOH | Pattern A |
| D3 | Neat | Predominantly amorphous |
| D4 | DCM | Predominantly amorphous |
| D5 | THF | Predominantly amorphous |
| D6 | TBME | Predominantly amorphous |

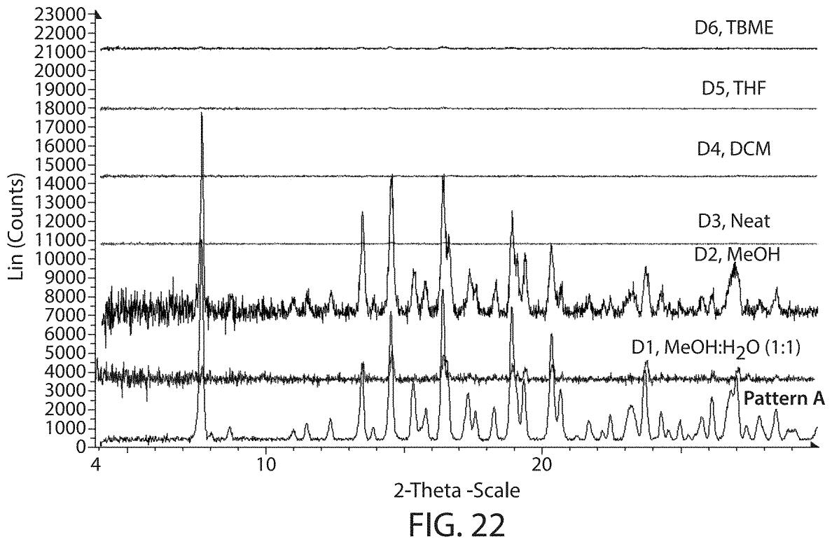

The XRPD analysis of neat and solvent drop grinding revealed the following results:
Grinding in MeOH produces Pattern A.
Grinding in MeOH:H₂O (1:1) produces semi-crystalline material.
Dry grinding and solvent drop grinding in the presence of DCM, TBME and THF results in produces predominantly amorphous with few peaks from Pattern A.
FIG. 22 illustrates the XRPD comparison of grinding experiments.

### Example 9: Vapor Diffusion Experiments of Compound 1.HCl

### Vapor Diffusion of Crystalline Compound 1.HCl

Vapor diffusion experiments of crystalline Compound 1.HCl were carried out by placing 20-25 mg of the salt in 4 mL vial and placing it in a 20 mL scintillation vial containing 2 mL of the solvent listed in Table 7. The scintillation vials were then placed in a well plate at 35° C. and analyzed by XRPD the following day. After the XRPD analysis, the vials with left over sample were placed in a vacuum oven and were analyzed by XRPD after two days.

TABLE 7
Summary of vapor diffusion experiments for crystalline Compound 1•HC1.

| | Solvent used for | Result after diffusion | Result after vacuum |
|---|---|---|---|
| Sample ID | diffusion | (XRPD analysis) | drying |
| E1 | MeOH | Pattern A | Pattern A |
| E2 | EtOH:H₂O (95:5) | Solid deliquesced | Pattern A |
| E3 | Acetone | Pattern A | Pattern A |
| E4 | DCM | Pattern A | Pattern A |
| E5 | IPA | Pattern A | Pattern A |
| E6 | THF | Pattern A | Pattern A |
| E7 | TBME | Pattern A | Pattern A |
| E8 | IPA:H₂O (9:1) | Pattern A | Pattern A |

### Vapor Diffusion of Amorphous Compound 1.HCl

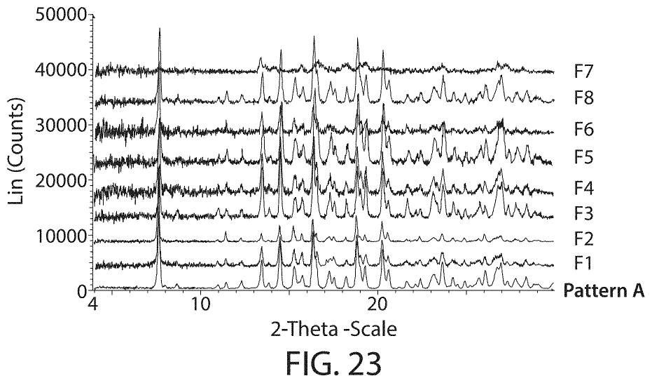

Similarly, vapor diffusion experiments for amorphous Compound 1.HCl were carried by placing 10-15 mg of the salt in 4 mL vial and placing it in a 20 mL scintillation vial containing 2 mL of the solvent listed in Table 8. The scintillation vials were left undisturbed at room temperature and were analyzed by XRPD the following day. XRPD analysis of the above samples revealed that the amorphous salt had transformed to Pattern A. Sample ID F7 however some amorphous content had in it (FIG. 23).

TABLE 8
Summary of vapor diffusion experiments for amorphous Compound 1•HCl.

| | Solvent used for | Result after diffusion |
|---|---|---|
| Sample ID | diffusion | (XRPD analysis) |
| Fl | MeOH | Pattern A |
| F2 | EtOH:H₂O (95:5) | Pattern A |
| F3 | Acetone | Pattern A |
| F4 | DCM | Pattern A |
| F5 | IPA | Pattern A |
| F6 | THF | Pattern A |
| F7 | TBME | Pattern A + amorphous salt |
| F8 | H₂O | Pattern A |

### Example 10: Relative Stability of the Forms

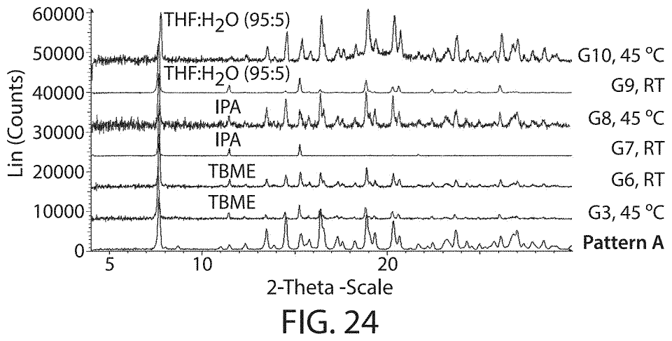

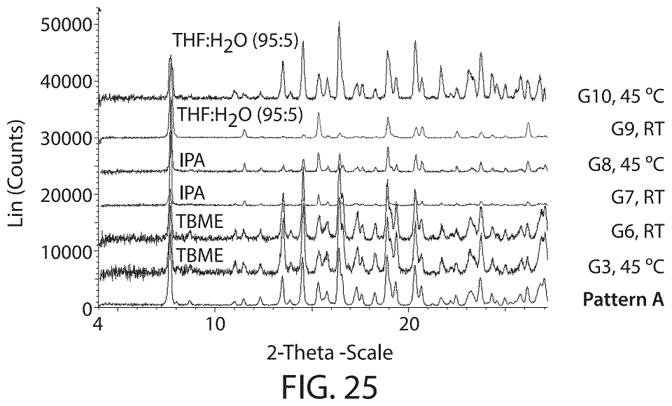

The crystalline Compound 1.HCl (Pattern A) and the amorphous were used for the competitive slurries at room temperature (RT) and 45° C. to determine the most stable form or solvate/hydrate formation in three different solvents (TBME, IPA and THF:H₂O (95:5) (Table 9). For the competitive slurries, saturated solutions of Pattern A in 0.5 mL of TBME, IPA and THF:H₂O (95:5) were prepared (two sets for each solvent). 15-20 mg of amorphous form and Pattern A were added to each vial and stirred at RT and 45° C. An aliquot from each slurries were drawn and analyzed by XRPD at t=5 min (FIG. 24). All the slurry samples after t=5 min transformed to Pattern A. Further analysis of the slurries after 24 h also did not exhibit any change in the crystalline form (Pattern A) as illustrated in FIG. 25.

TABLE 9
Competitive slurries of Compound 1•HCl.

| Solvent | Sample ID: 45° C. | Sample ID: RT |
|---|---|---|
| TBME | G3 | G6 |
| IPA | G8 | G7 |
| THF:H₂O (95:5) | G10 | G9 |

### Example 11: XRPD Peak Identification of Compound 1.HCl, Pattern A

An exemplary XRPD pattern of Pattern A is shown in FIG. 26. Table 10 shows the listing of exemplary peaks of the XRPD pattern of FIG. 26.

TABLE 10
Peak list for Pattern A XRPD.

| Angle (2θ) degree | Intensity % | d value (Å) |
|---|---|---|
| 7.64 | 100 | 11.566 |
| 12.32 | 10.9 | 7.180 |
| 13.45 | 39.2 | 6.578 |
| 14.49 | 64.3 | 6.106 |
| 15.39 | 25.4 | 5.751 |
| 16.46 | 65.3 | 5.380 |
| 17.37 | 23.5 | 5.101 |
| 18.25 | 16.8 | 4.857 |
| 18.97 | 53.4 | 4.674 |
| 20.40 | 44.2 | 4.349 |
| 23.69 | 36.8 | 3.753 |
| 24.34 | 14.4 | 3.654 |
| 26.05 | 16.7 | 3.418 |
| 26.89 | 27.8 | 3.313 |
| 28.45 | 16.9 | 3.135 |
| 35.64 | 11.3 | 2.517 |

### Example 12: Efficacy Study of Compound 1 in a Hound Cardiopulmonary Bypass Model

The objective of this study was to demonstrate the efficacy of Compound 1 compared to the Standard of Care (SOC), heparin, for preventing activation of blood coagulation components while using the Cardiopulmonary Bypass (CPB) circuit during an extended run time on Day 1 in a mixed breed hound dog model. The study design is shown in Table 11:

TABLE 11
Experimental Design (Target Doses of Compound 1^{b})

| | | IV | IV Bolus | | IV | Dose | Dose | |
|---|---|---|---|---|---|---|---|---|
| | | Loading | Dose | Dose | Infusion | Infusion | Concentration | Compound |
| | No. of | Dose | Volume | Concentration | Dose Level | Rate | for IV | 1 in Prime |
| Group | Animals | (mg/kg) | (mL/kg) | for IV Bolus | (mg/kg/hr) | (mL/kg/hr) | Infusion | Solution |
| 1 | 3 | NA | NA | NA | 3 | 5 | 0.6 mg/mL | NA |
| 2 | 2 | 3 | 1 | 0.6 μg/mL and | 3 | 5 | 0.6 μg/mL | 0.01 mg/mL |
| | | | | 3 mg/mL^{a} | | | | |
| 3 | 2 | 10 | 1 | 10 mg/mL | 10 | 5 | 2.0 mg/mL | 0.01 mg/mL |
| 4 | 2 | 10 | 1 | 10 mg/mL | 10 | 5 | 2.0 mg/mL | 0.01 mg/mL |
| 5 | 2 | 10 | 1 | 10 mg/mL | 10 | 5 | 2.0 mg/mL | 0.01 mg/mL |

NA—Not Applicable
^{a}Animal No. 1001 received 0.6 μg/mL and Animal No. 1004 received 3 mg/mL.
^{b}Doses shown are targets for the dosing on this study; actual dose values are shown in the results section.

The following parameters and endpoints were evaluated in this study: mortality, body weight, physical, clinical pathology parameters (hematology and coagulation), coagulation time, and bioanalytical parameters.

### Experimental Design

### Administration

The vehicle and test article were administered via intravenous (IV) infusion once on Day 1 for 135 minutes (initiated 30 minutes prior to starting the Cardiopulmonary Bypass (CPB) and continuing for 105 minutes of CPB). Group 2 animals received a 0.6 μg/mL or 3.0 mg/mL IV bolus dose immediately prior to the start of IV infusion. Group 3, 4, and 5 animals received a 10 mg/kg IV bolus dose prior to the start of the IV infusion; with the CPB machine primed with test article at 10 μg/mL.

### Surgical Procedure

Group 1 had an infusion pump setup with an open system/reservoir. Infusion of the Compound 1 was started 30 minutes prior to the animal being placed on the CPB pump. The CPB pump was primed with 0.9% saline.

Groups 2, 3, and 4 had an infusion pump setup with an open system/reservoir. Venous and arterial sheaths were flushed with the Compound 1 at a concentration of 10 μg/mL. An IV bolus dose of the test article was administered immediately prior to the start of the infusion. Infusion of Compound 1 was started 30 minutes prior to the animal being placed on the CPB pump. The CPB patient was primed with 10 μg/mL of the Compound 1 prior to initiation of the CPB pump.

Group 5 had an infusion pump setup with a closed system/“bag.” Venous and arterial sheaths were then flushed with Compound 1 at 10 μg/mL. An IV bolus dose of the Compound 1 was administered immediately prior to the start of the infusion. Infusion of Compound 1 was started 30 minutes prior to the animal being placed on the CPB pump.

### Results

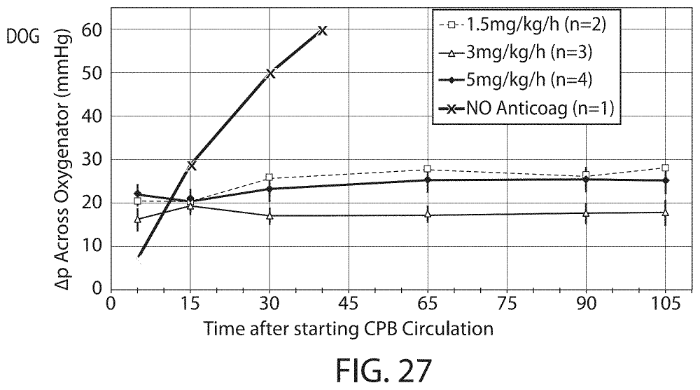

FIG. 27 shows pressure gradients assessed across the membrane oxygenator. Studies previously conducted with no anticoagulant demonstrated that the pressure across the membrane oxygenator built within 15 minutes of pump start and exponentially increased over the next 30 minutes such that the oxygenator was occluded and the circulation was stopped, whereas with Compound 1 at multiple doses, the pressure gradient across the membrane oxygenator stayed consistent through the entire run, indicating that the test article successfully maintained anticoagulation allowing the continuation of the pump run for the entirety of the protocol.

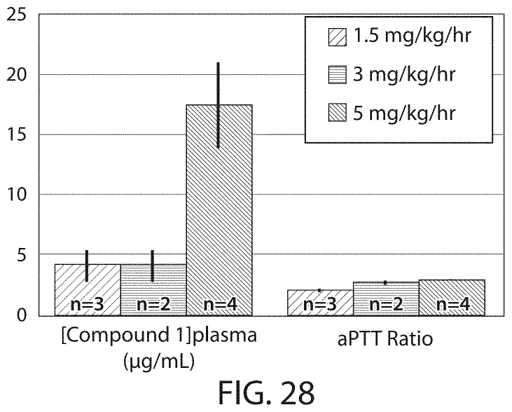

FIG. 28 shows a correlation between Compound 1 plasma concentration and aPTT. All animals survived to study termination. Overall, Compound 1 was not associated with any increases in morbidity or mortality at the dose levels used in this study during the Cardiopulmonary bypass/ECMO protocol.

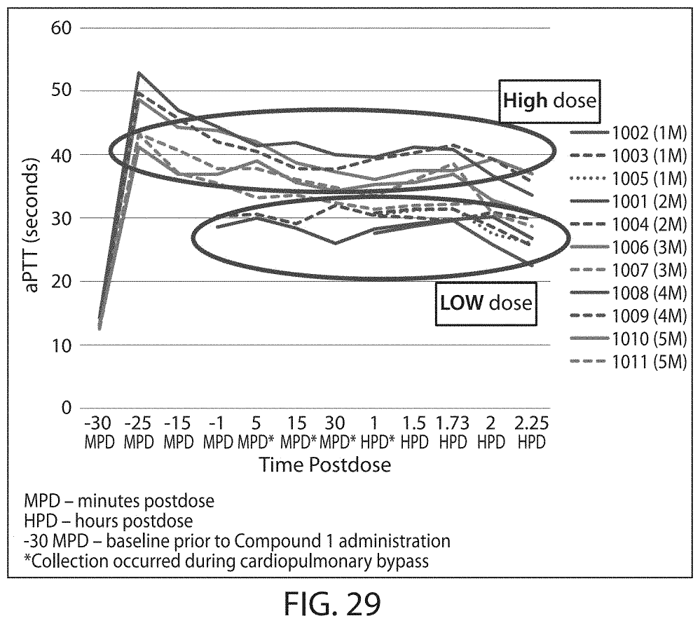

During Compound 1 infusion and prior to CPB, aPTT was moderately to markedly prolonged in all animals (FIG. 29). Prolongations in aPTT persisted throughout Compound 1 infusion and CPB. In groups that received a loading dose of Compound 1 (Groups 2 through 5), prolongations in aPTT were most pronounced prior to (Group 3 through 5) or during the first 30 minutes of CPB (Group 2), but then improved slightly before reaching steady-state. Group 1 animals did not receive an Compound 1 loading dose, and prolongations in aPTT remained relatively consistent at all measured timepoints during Compound 1 infusion in this group. In all groups following cessation of Compound 1 infusion and CPB, aPTT trended towards baseline values, but remained moderately prolonged at the conclusion of the study.

### CONCLUSIONS

Administration of the Compound 1 to the model was successful in preventing the activation of blood coagulation in components of cardiopulmonary bypass. The anticoagulant effects of Compound 1 were selective to inhibition of activated partial thromboplastin time (aPTT). Additionally, the data demonstrated that adding a bolus dose immediately prior to starting the infusion enabled targeted plasma levels of Compound 1 to rapidly be achieved, along with desired steady state levels, and was sufficient to achieve a successful 105-minute CPB run and prevent coagulation in most of the circuit components. Overall, these data indicate that Compound 1 may be an acceptable alternative to heparin in preventing blood coagulation in components of cardiopulmonary bypass.

### Equivalents

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A crystalline pharmaceutically acceptable salt of Formula (I)

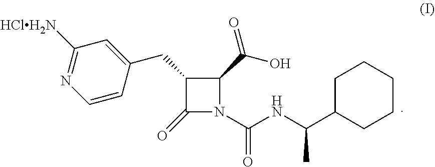

2. The crystalline pharmaceutically acceptable salt of claim 1, having an XRPD pattern with characteristic peaks between and including the following values of 2θ in degrees: 7.4 to 7.8, 13.3 to 13.7, 14.3 to 14.7, 15.2 to 15.6, 16.3 to 16.7, 17.2 to 17.6, 18.8 to 19.2, 20.2 to 20.6, 23.5 to 23.9, and 26.7 to 27.1.

3. The crystalline pharmaceutically acceptable salt of claim 1, having an XRPD pattern with characteristic peaks at the following values of 2θ in degrees: 7.6, 13.5, 14.5, 15.4, 16.5,

17. 4, 19.0, 20.4, 23.7, and 26.9.

4. The crystalline pharmaceutically acceptable salt of claim 1, having an XRPD pattern with characteristic peaks between and including the following values of 2θ in degrees: 7.4 to 7.8, 14.3 to 14.7, 16.3 to 16.7, 18.8 to 19.2, and 20.2 to 20.6.

5. The crystalline pharmaceutically acceptable salt of claim 1, having an XRPD pattern with characteristic peaks at the following values of 2θ in degrees: 7.6, 14.5, 16.5, 19.0, and 20.4.

6. The crystalline pharmaceutically acceptable salt of claim 1, having an XRPD pattern substantially as depicted in FIG. 1.

7. The crystalline pharmaceutically acceptable salt of claim 1, having an XRPD pattern substantially as depicted in FIG. 26.

8. The crystalline pharmaceutically acceptable salt of claim 1, wherein the crystalline pharmaceutically acceptable salt melts at a Tₒₙₛₑₜ from about 178° C. to about 192° C. as determined by DSC at a ramp rate of 10° C./min.

9. The crystalline pharmaceutically acceptable salt of claim 1, having a DSC thermogram substantially as depicted in FIG. 6.

10. An amorphous pharmaceutically acceptable salt of Formula (I)

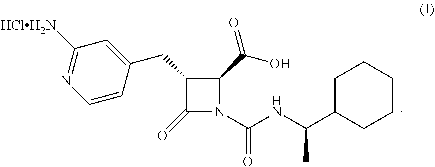

11. The amorphous pharmaceutically acceptable salt of claim 10, having an endotherm at a Tₒₙₛₑₜ from about 95° C. to about 105° C. as determined by DSC at a ramp rate of 10° C./min.

12. The amorphous pharmaceutically acceptable salt of claim 10, having a DSC thermogram substantially as depicted in FIG. 14.

13. The amorphous pharmaceutically acceptable salt of claim 10, wherein the amorphous pharmaceutically acceptable salt, when subjected to a temperature of about 140° C., transforms into the crystalline compound of claim 1 as indicated by DSC at a ramp rate of 10° C./min.

14. A pharmaceutical composition comprising a crystalline pharmaceutically acceptable salt of Formula (I) and a pharmaceutically acceptable excipient.

15. A pharmaceutical composition comprising an amorphous pharmaceutically acceptable salt of Formula (I) and a pharmaceutically acceptable excipient.

16. A method of treating deep vein thrombosis in a subject that has suffered an ischemic event, comprising administering to the subject an effective amount of a compound of any one of claims 1-13 or a pharmaceutical composition of claim 14 or 15.

17. A method of treating a subject that has edema, comprising administering to the subject an effective amount of a compound of any one of claims 1-13 or a pharmaceutical composition of claim 14 or 15.

18. A method of treating a thromboembolic disorder in a subject in need thereof, the method comprising administering to the subject an effective amount of a compound represented by 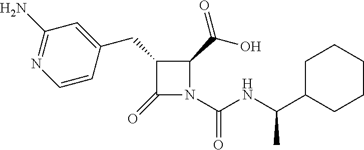 [SMILES: C[C@@H](NC(=O)N1C(=O)[C@H](Cc2ccnc(N)c2)[C@H]1C(=O)O)C1CCCCC1]
or a pharmaceutically acceptable salt thereof, wherein the subject is exposed to an artificial surface.

19. A method of reducing the risk of a thromboembolic disorder in a subject in need thereof, the method comprising administering to the subject an effective amount of a compound represented by 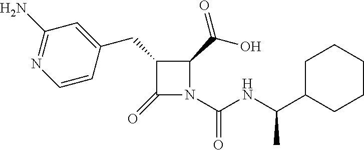 [SMILES: C[C@@H](NC(=O)N1C(=O)[C@H](Cc2ccnc(N)c2)[C@H]1C(=O)O)C1CCCCC1]
or a pharmaceutically acceptable salt thereof, wherein the subject is exposed to an artificial surface.

20. A method of prophylaxis of a thromboembolic disorder in a subject in need thereof, the method comprising administering to the subject an effective amount of a compound represented by 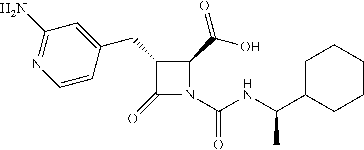 [SMILES: C[C@@H](NC(=O)N1C(=O)[C@H](Cc2ccnc(N)c2)[C@H]1C(=O)O)C1CCCCC1]
or a pharmaceutically acceptable salt thereof, wherein the subject is exposed to an artificial surface.

21. The method of any one of claims 18-20, wherein the artificial surface is in contact with blood in the subject's circulatory system.

22. The method of any one of claims 18-21, wherein the artificial surface is an implantable device, a dialysis catheter, a cardiopulmonary bypass circuit, an artificial heart valve, a ventricular assist device, a small caliber graft, a central venous catheter, or an extracorporeal membrane oxygenation (ECMO) apparatus.

23. The method of any one of claims 18-22, wherein the artificial surface causes or is associated with the thromboembolic disorder.

24. The method of any one of claims 18-23, wherein the thromboembolic disorder is a venous thromboembolism, deep vein thrombosis, or pulmonary embolism.

25. The method of any one of claims 18-24, wherein the thromboembolic disorder is a blood clot.

26. The method of any one of claims 18-25, further comprising conditioning the artificial surface with a separate dose of the compound or pharmaceutically acceptable salt thereof, prior to contacting the artificial surface with blood in the circulatory system of the subject.

27. The method of any one of claims 18-26, further comprising conditioning the artificial surface with a separate dose of the compound or pharmaceutically acceptable salt thereof prior to or during administration of the compound or a pharmaceutically acceptable salt thereof to the subject.

28. The method of any one of claims 18-27, further comprising conditioning the artificial surface with a separate dose of the compound or pharmaceutically acceptable salt thereof prior to and during administration of the compound or a pharmaceutically acceptable salt thereof to the subject.

29. A method of treating the blood of a subject in need thereof, the method comprising administering to the subject an effective amount of a compound represented by 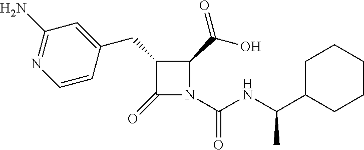 [SMILES: C[C@@H](NC(=O)N1C(=O)[C@H](Cc2ccnc(N)c2)[C@H]1C(=O)O)C1CCCCC1]
or a pharmaceutically acceptable salt thereof.

30. A method of maintaining the plasma level of a compound represented by 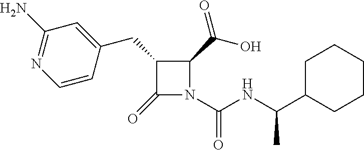 [SMILES: C[C@@H](NC(=O)N1C(=O)[C@H](Cc2ccnc(N)c2)[C@H]1C(=O)O)C1CCCCC1]
or a pharmaceutically acceptable salt thereof, in the blood of a subject in contact with an artificial surface, the method comprising:
(i) administering the compound or pharmaceutically acceptable salt thereof to the subject prior to or while contacting the artificial surface with the blood of the subject; and
(ii) conditioning an artificial surface with the compound or a pharmaceutically acceptable salt thereof prior to or while contacting the artificial surface with the blood of the subject;
thereby maintaining the plasma level of the compound or a pharmaceutically acceptable salt thereof in the blood of the subject.

31. The method of claim 30, wherein the compound, or a pharmaceutically acceptable salt thereof, maintains a constant activated partial thromboplastin time (aPTT) in the blood of the subject before and after contact with the artificial surface.

32. The method of claim 30 or 31, wherein the compound or a pharmaceutically acceptable salt thereof is administered to the subject prior to and while contacting the artificial surface with the blood of the subject.

33. The method of any one of claims 30-32, wherein the artificial surface is conditioned with the compound or a pharmaceutically acceptable salt thereof prior to and while contacting the artificial surface with the blood of the subject.

34. The method of any one of claims 29-33, wherein the method further prevents or reduces risk of a blood clot formation in the blood of the subject in contact with the artificial surface.

35. The method of any one of claims 29-34, wherein the artificial surface is a cardiopulmonary bypass circuit.

36. The method of any one of claims 29-34, wherein the artificial surface is an extracorporeal membrane oxygenation (ECMO) apparatus.

37. The method of claim 36, wherein the ECMO apparatus is venovenous ECMO apparatus or venoarterial ECMO apparatus.

38. A method of preventing or reducing a risk of a thromboembolic disorder in a subject during or after a medical procedure, comprising:
(i) administering to the subject an effective amount of a compound represented by: 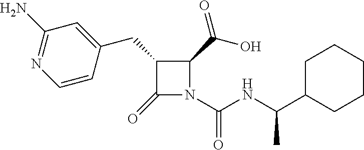 [SMILES: C[C@@H](NC(=O)N1C(=O)[C@H](Cc2ccnc(N)c2)[C@H]1C(=O)O)C1CCCCC1]
or pharmaceutically acceptable salt thereof, before, during, or after the medical procedure; and
(ii) contacting blood of the subject with an artificial surface;
thereby preventing or reducing the risk of the thromboembolic disorder during or after the medical procedure.

39. The method of claim 38, wherein the artificial surface is conditioned with the compound or pharmaceutically acceptable salt thereof prior to administration of the compound to the subject prior to, during, or after the medical procedure.

40. The method of claim 38 or 39, wherein the artificial surface is conditioned with a solution comprising the compound or a pharmaceutically acceptable salt thereof prior to administration of the compound or a pharmaceutically acceptable salt thereof to the subject prior to, during, or after the medical procedure.

41. The method of claim 40, wherein the solution is a saline solution, Ringer's solution, or blood.

42. The method of any one of claims 38-41, wherein the thromboembolic disorder is a blood clot.

43. The method of any one of claims 38-42, wherein the medical procedure comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

44. The method of any one of claims 38-43, wherein the medical procedure comprises a cardiopulmonary bypass.

45. The method of any one of claims 38-43, wherein the medical procedure comprises an oxygenation and pumping of blood via extracorporeal membrane oxygenation (ECMO).

46. The method of claim 45, wherein the ECMO is venovenous ECMO or venoarterial ECMO.

47. The method of any one of claims 16-46, wherein the pharmaceutically acceptable salt of the compound is a hydrochloride salt.

48. The method of any one of claims 16-47, wherein the compound is administered to the subject intravenously.

49. The method of any one of claims 16-47, wherein the compound is administered to the subject subcutaneously.

50. The method of any one of claims 16-47, wherein the compound is administered to the subject as a continuous intravenous infusion.

51. The method of any one of claims 16-47, wherein the compound is administered to the subject as a bolus.

52. The method of any one of claims 16-51, wherein the subject is a human.

53. The method of any one of claims 16-52, wherein the subject has an elevated risk of a thromboembolic disorder.

54. The method of claim 53, wherein the thromboembolic disorder is a result of a complication in surgery.

55. The method of any one of claims 16-54, wherein the subject is sensitive to or has developed sensitivity to heparin.

56. The method of any one of claims 16-55, wherein the subject is resistant to or has developed resistance to heparin.

57. The method of any one of claims 18-56, wherein the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

58. The method of any one of claims 18-57, wherein the subject is a pediatric subject.

59. The method of any one of claims 18-57, wherein the subject is an adult.
